SEQUENCE LISTING (29 sequences)

SEQ ID NO: 1 - protein, 8 aa, synthetic construct
  REGION 1..8: note = CDR sequence
GFTFNTYA

SEQ ID NO: 2 - protein, 10 aa, synthetic construct
  REGION 1..10: note = CDR sequence
IRSKYNNYAT

SEQ ID NO: 3 - protein, 16 aa, synthetic construct
  REGION 1..16: note = CDR sequence
VRHGNFGNSYVSWFAY

SEQ ID NO: 4 - protein, 9 aa, synthetic construct
  REGION 1..9: note = CDR sequence
TGAVTTSNY

SEQ ID NO: 5 - protein, 9 aa, synthetic construct
  REGION 1..9: note = CDR sequence
ALWYSNLWV

SEQ ID NO: 6 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, synthetic construct
  REGION 1..8: note = CDR sequence
GFTFHDYA

SEQ ID NO: 9 - protein, 8 aa, synthetic construct
  REGION 1..8: note = CDR sequence
ISWNSGTI

SEQ ID NO: 10 - protein, 15 aa, synthetic construct
  REGION 1..15: note = CDR sequence
AKDIQYGNYYYGMDV

SEQ ID NO: 11 - protein, 6 aa, synthetic construct
  REGION 1..6: note = CDR sequence
QSVSSY

SEQ ID NO: 12 - protein, 9 aa, synthetic construct
  REGION 1..9: note = CDR sequence
QQRSNWPIT

SEQ ID NO: 13 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 18 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 19 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 20 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 25 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 27 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

(19) United States
(12) Patent Application Publication
(10) Pub. No.: US 2024/0174761 A1
(43) Pub. Date: May 30, 2024

(54) BISPECIFIC ANTIBODIES AGAINST CD3 AND CD20 FOR TREATING RICHTER'S SYNDROME

(71) Applicant: GENMAB A/S, Valby (DK)

(72) Inventors: Brian ELLIOTT, Hoboken, NJ (US); Rebecca X.M.S. VALENTIN, Copenhagen N (DK); Daniela HOEHN, Arverne, NY (US); Marcia RIOS, Sayreville, NJ (US)

(21) Appl. No.: 18/500,673

(22) Filed: Nov. 2, 2023

Related U.S. Application Data
(60) Provisional application No. 63/421,766, filed on Nov. 2, 2022.
(60) Provisional application No. 63/430,546, filed on Dec. 6, 2022.

(51) Int. Cl. C07K 16/28; A61P 35/00; A61K 39/00

(52) U.S. Cl. CPC C07K 16/2887; A61P 35/00; C07K 16/2809; A61K 2039/505; A61K 2039/545; C07K 2317/21; C07K 2317/24; C07K 2317/31

(57) ABSTRACT

Provided are methods of clinical treatment of Richter's syndrome in human subjects using a bispecific antibody which binds to CD3 and CD20.

### FIELD

The present invention relates to the use of bispecific antibodies targeting both CD3 and CD20 in the treatment of Richter's syndrome (RS). Advantageous treatment regimens are also provided.

### BACKGROUND

Chronic lymphocytic leukemia (CLL) is a B-cell malignancy that originates from uncontrolled proliferation of immature lymphocytes in the bone marrow and involves circulating tumor cells in the blood. CLL is characterized by accumulation of clonal CD5+CD19+CD20+CD23+B cells in the bone marrow, blood, and lymphoid organs such as lymph nodes and spleen (Zenz et al., Nat Rev Cancer 2010; 10:37-50). CLL is often a slow-growing cancer. CLL is primarily a disease of older adults, with a median age of 70 years at the time of diagnosis. CLL is the most common leukemia in adults in Western countries, accounting for approximately 25% to 30% of all leukemias in the US with estimated 20,720 new cases and 3,930 deaths (Siegel et al., CA Cancer J Clin 2019; 69:7-34). Worldwide, there are approximately 105,000 cases per year, of which 35,000 are deaths (Global Burden of Disease Cancer, Fitzmaurice et al., JAMA Oncol 2018; 4:1553-68).

In contrast, lymphoma originates from uncontrolled proliferation of lymphocytes in organs outside of the bone marrow. Although in some lymphomas, bone marrow can also have tumor cell infiltrates. Lymphoma cells will usually not appear in the peripheral blood.

Richter's syndrome (RS), also called Richter's transformation (RT), is the development of an aggressive lymphoma arising in the background of CLL or SLL (small lymphocytic lymphoma) (Swerdlow et al., 2017; WHO Classification of Tumours of Haematopoietic and Lymphoid Tissues. International Agency for Research on Cancer, Lyon, France). The annual incidence rate of RS in patients with CLL has been estimated around 0.5% to 1%, with an overall incidence rate in approximately 5% to 16% of all patients with CLL (Rossi et al., Br J Haematol, 2008; 142, 202-215). In a more recent study, Richter's Syndrome was found to occur in approximately 2-10% of all CLL/SLL patients during the course of their disease (Wang Y, et al. Haematologica. 2020; 105:765-73). Approximately 90% of the time, RS presents as diffuse large B-cell lymphoma (RS-DLBCL)(Parikh et al., Br J Haematol, 2013; 162, 774-782; Rossi et al., Br J Haematol, 2008; 142, 202-215). Approximately eighty percent of RS-DLBCL are clonally related to the original CLL, which is historically chemorefractory, with a median survival of 12 months (Eyre et al., Br J Haematol, 2016; 175, 43-54.; Langerbeins et al., Am J Hematol 89, 2014; E239-243; Rogers et al., Br J Haematol, 2018; 180, 259-266; Tsimberidou et al., Clin Lymphoma Myeloma Leuk, 2013; 13, 568-574). Monotherapy using novel agents such as BTK inhibitor and BCL2 inhibitor has done little to change the outcome of RS. Among 29 patients with RS-DLBCL treated with acalabrutinib, ORR, median duration of response, and median PFS were 38%, 5 months, and 3 months, respectively (Hillmen et al., Blood, 2016; 128, 60-60). In 7 patients with RS-DLBCL, venetoclax achieved an ORR of 43% with unknown duration. Checkpoint inhibitors such as pembrolizumab and nivolumab, either as monotherapy or in combination with BTK inhibitor, have demonstrated ORR ranging from 40% to 60% with a relatively short PFS of 4 months (Ding et al., Blood 2017; 129, 3419-3427; Jain et al., Blood 2016; 128, 59-59; Younes et al., Blood 2017; 130, 833-833). Due to the short duration of response to the chemoimmunotherapy, autologous and allogeneic stem cell transplantation has been used as post-induction therapy to prolong the survival for fit/young RS patients. However, approximately 80% to 90% of RS patients are ineligible for receiving a transplant due to: 1) being unable to achieve a remission by the induction therapy; 2) comorbidities or age. In summary, in the era of novel agents, the incidence of RS has not decreased and the prognosis of subjects with RS remains poor.

Hence, there remains an unmet need with respect to treatment options for patients with Richter's syndrome.

### SUMMARY

Provided herein are methods of treating human subjects presenting with Richter's syndrome by administering a bispecific antibody which binds to CD3 and CD20 and, in particular, advantageous clinical treatment regimens.

In one aspect, provided herein is a method of treating Richter's syndrome in a human subject, the method comprising administering (e.g., subcutaneously) to the subject an effective amount of a bispecific antibody (e.g., epcoritamab) comprising:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose ranging from 12-60 mg in 28-days cycles. In some embodiments, the bispecific antibody is administered at a dose of (or a dose of about) 12 mg, 24 mg, 48 mg, or 60 mg. In some embodiments, the bispecific antibody is administered at a dose of (or a dose of about) 12 mg. In some embodiments, the bispecific antibody is administered at a dose of (or a dose of about) 24 mg. In some embodiments, the bispecific antibody is administered at a dose of (or a dose of about) 48 mg. In some embodiments, the bispecific antibody is administered at a dose of (or a dose of about) 60 mg.

In some embodiments, the bispecific antibody is administered once every week, e.g., for 2.5 28-day cycles (i.e., days 15 and 22 of cycle 1, and days 1, 8, 15, and 22 of cycles 2-3). In some embodiments, the bispecific antibody is administered once every two weeks after the weekly administration, e.g., for six 28-day cycles. In some embodiments, the bispecific antibody is administered once every four weeks after the biweekly administration. In a further embodiment, a priming dose (e.g., 0.05-0.35 mg, for example, 0.16 mg or about 0.16 mg) of the bispecific antibody is administered two weeks prior to administering the weekly dose of 24 mg or 48 mg. In a further embodiment, the priming dose is administered one week before the intermediate dose, and the intermediate dose is administered one week before the first weekly dose of 24 mg or 48 mg.

In some embodiments, the bispecific antibody is administered in 28-day cycles, wherein:
a) in cycle 1, a priming dose (e.g., 0.05-0.35 mg, for example, 0.16 mg or about 0.16 mg) is administered on day 1, an intermediate dose (e.g., 0.6-1.2 mg, for example, 0.8 mg or about 0.8 mg) on day 8, and a full dose of 12-60 mg (e.g., 12 mg, 24 mg, 48 mg, or 60 mg) on days 15 and 22;
b) in cycles 2-3, a full dose of 12-60 mg (e.g., 12 mg, 24 mg, 48 mg, or 60 mg) is administered on days 1, 8, 15, and 22;
c) in cycles 4-9, a full dose of 12-60 mg (e.g., 12 mg, 24 mg, 48 mg, or 60 mg) is administered on days 1 and 15; and
d) in cycle 10 and subsequent cycles, a full dose of 12-60 mg (e.g., 12 mg, 24 mg, 48 mg, or 60 mg) is administered on day 1.

In a further embodiment, the subject has refractory and/or relapsed Richter's syndrome after receiving the two prior antineoplastic therapies.

In some embodiments, the subject is treated with prophylaxis for cytokine release syndrome (CRS). In some embodiments, the prophylaxis comprises administering a corticosteroid (e.g., prednisolone at a dose of, e.g., 100 mg or equivalent thereof, including oral dose) on, for example, the same day as the bispecific antibody. In some embodiments, the corticosteroid is further administered on the second, third, and fourth days after administering the bispecific antibody.

In some embodiments, the subject is administered premedication, such as antihistamine (e.g., diphenhydramine, intravenously or orally at a dose of, e.g., 50 mg or equivalent thereof) and/or antipyretic (e.g., acetaminophen at a dose of, e.g., 560-1000 mg), to reduce reactions to injections. In some embodiments, the premedication is administered on the same day as the bispecific antibody.

In some embodiments, the prophylaxis and premedication are administered during cycle 1. In some embodiments, the prophylaxis is administered during cycle 2 when the subject experiences CRS greater than grade 1 after the last administration of the bispecific antibody in cycle 1. In some embodiments, the prophylaxis is continued in a subsequent cycle, when in the last administration of the bispecific antibody of the previous cycle, the subject experiences CRS greater than grade 1. In a further embodiment, the premedication is administered during cycle 2. In a further embodiment, the premedication is administered during subsequent cycles.

In some embodiments, the subject is treated with antipyretics and hydration if the subject develops Grade 1 CRS. In some embodiments, the subject is treated with tocilizumab and/or dexamethasone or its equivalent of methylprednisolone if the subject develops Grade 2 CRS. In some embodiments, the subject is treated with tocilizumab and dexamethasone (e.g., at a dose of 10-20 mg or its equivalent of methylprednisolone, e.g., administered once every 6 hours) if the subject develops Grade 3 CRS. In a further embodiment, the subject is treated with tocilizumab and methylprednisolone (e.g., at a dose of 1000 mg/day) if the subject develops Grade 4 CRS. In a further embodiment, tocilizumab is switched to siltuximab if the subject does not respond to tocilizumab.

In some embodiments, the subject is administered prophylaxis for tumor lysis syndrome (TLS). In some embodiments, the prophylaxis for TLS comprises administering one or more uric acid reducing agents prior to administration of the bispecific antibody. In some embodiments, allopurinol and rasburicase are administered as the uric acid reducing agents. In a further embodiment, allopurinol is administered at least 72 hours prior to administration of the bispecific antibody. In a further embodiment, rasburicase is administered after administering allopurinol and prior to administering the bispecific antibody. In some embodiments, when a subject shows signs of TLS, supportive therapy, such as rasburicase and/or allopurinol, may be used.

In some embodiments, the subject treated with the methods described herein achieves a complete response, a partial response, or stable disease, e.g., as defined by Lugano criteria (Cheson et al., 2014).

In some embodiments, the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the first antigen-binding region of the bispecific antibody comprises a VH region comprising the amino acid sequence of SEQ ID NO: 6, and the VL region comprising the amino acid sequence of SEQ ID NO: 7; and the second antigen-binding region comprises a VH region comprising the amino acid sequence of SEQ ID NO: 13, and the VL region comprising the amino acid sequence of SEQ ID NO: 14.

In some embodiments, the first binding arm of the bispecific antibody is derived from a humanized antibody, preferably from a full-length IgG1,λ (lambda) antibody. In some embodiments, the second binding arm of the bispecific antibody is derived from a human antibody, preferably from a full-length IgG1,κ (kappa) antibody. In some embodiments, the bispecific antibody is a full-length antibody with a human IgG1 constant region.

In some embodiments, the bispecific antibody comprises an inert Fc region, for example, an Fc region in which the amino acids in the positions corresponding to positions L234, L235, and D265 in the human IgG1 heavy chain constant region of SEQ ID NO: 15 are F, E, and A, respectively. In some embodiments, the bispecific antibody comprises substitutions which promote bispecific antibody formation, for example, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In some embodiments, the bispecific antibody has both an inert Fc region (e.g., substitutions at L234, L235, and D265 (e.g., L234F, L235E, and D265A)) and substitutions which promote bispecific antibody formation (e.g., F405L and K409R). In a further embodiment, the bispecific antibody comprises heavy chain constant regions comprising the amino acid sequences of SEQ ID NOs: 19 and 20.

In some embodiments, the bispecific antibody comprises a first heavy chain and a first light chain comprising (or consisting of) the amino acid sequences set forth in SEQ ID NOs: 24 and 25, respectively, and a second heavy chain and a second light chain comprising (or consisting of) the amino acid sequences set forth in SEQ ID NOs: 26 and 27, respectively. In some embodiments, the bispecific antibody is epcoritamab, or a biosimilar thereof.

### FIGURES

FIG. 1: Results from Phase 1b/2 Trial: Treatment-emergent adverse events; data cutoff: Sep. 8, 2022.

FIG. 2: Results from Phase 1b/2 Trial: CRS events by dosing period; data cutoff: Sep. 8, 2022.

FIG. 3: Results from Phase 1b/2 Trial: Depth and duration of the responses; data cut-off Sep. 8, 2022.

FIG. 4: Results from Phase 1b/2 Trial; Tumor reduction from baseline; data cutoff: Sep. 8, 2022.

FIG. 5: Clinical case study (RS-DLBCL. A: Baseline PET/CT scan, mesenteric mass: 11.6×7.2 cm, standardized uptake value (SUV) max 6.3 in abdominal/pelvic mass; B: 12-week PET/CT scan, mesenteric mass: 0.5×0.5 cm, SUV max 2.0 in abdominal pelvic mass; C: 76-week PET/CT scan, mesenteric mass 0.5×0.5 cm, background uptake only.

### DETAILED DESCRIPTION

### Definitions

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized (see, e.g., Fundamental Immunology Ch. 7 (Paul, W., ed., 2nd ed. Raven Press, N.Y. (1989)). Briefly, each heavy chain typically is comprised of a heavy chain variable region (abbreviated herein as VH or V_{H}) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J Mol Biol 1987; 196:90117). Unless otherwise stated or contradicted by context, CDR sequences herein are identified according to IMGT rules (Brochet X., Nucl Acids Res 2008; 36:W503-508; Lefranc M P., Nucl Acids Res 1999; 27:209-12; www.imgt.org/). Unless otherwise stated or contradicted by context, reference to amino acid positions in the constant regions is according to the EU-numbering (Edelman et al., PNAS. 1969; 63:78-85; Kabat et al., Sequences of Proteins of Immunological Interest, Fifth Edition. 1991 NIH Publication No. 91-3242). For example, SEQ ID NO: 15 sets forth amino acids positions 118-447, according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is within the ability of one of ordinary skill in the art to align a sequence or segment in a sequence and thereby determine the corresponding position in a sequence to an amino acid position according to the present invention.

The term “antibody” (Ab) as used herein in the context of the present invention refers to an immunoglobulin molecule which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term antibody, unless specified otherwise, also encompasses polyclonal antibodies, monoclonal antibodies (mAbs), antibody-like polypeptides, chimeric antibodies and humanized antibodies. An antibody as generated can possess any isotype.

The term “antibody fragment” or “antigen-binding fragment” as used herein refers to a fragment of an immunoglobulin molecule which retains the ability to specifically bind to an antigen, and can be generated by any known technique, such as enzymatic cleavage, peptide synthesis, and recombinant techniques. Examples of antibody fragments include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 1989; 341: 54446), which consists essentially of a VH domain and also called domain antibodies (Holt et al; Trends Biotechnol 2003; 21:484-90); (vi) camelid or nanobodies (Revets et al; Expert Opin Biol Ther 2005; 5:111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see, e.g., Bird et al., Science 1988; 242.42326 and Huston et al., PNAS 1988; 85:587983). Such single chain antibodies are encompassed within the term antibody fragment unless otherwise noted or clearly indicated by context.

The term “antibody-binding region” or “antigen-binding region” as used herein refers to the region which interacts with the antigen and comprises both the VH and the VL regions. The term antibody when used herein refers not only to monospecific antibodies, but also multispecific antibodies which comprise multiple, such as two or more, e.g., three or more, different antigen-binding regions. The term antigen-binding region, unless otherwise stated or clearly contradicted by context, includes fragments of an antibody that are antigen-binding fragments, i.e., retain the ability to specifically bind to the antigen.

As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g., IgG1, is mentioned, the term is not limited to a specific isotype sequence, e.g., a particular IgG1 sequence, but is used to indicate that the antibody is closer in sequence to that isotype, e.g. IgG1, than to other isotypes. Thus, e.g., an IgG1 antibody may be a sequence variant of a naturally-occurring IgG1 antibody, which may include variations in the constant regions.

The term “bispecific antibody” or “bs” or “bsAb” as used herein refers to an antibody having two different antigen-binding regions defined by different antibody sequences. A bispecific antibody can be of any format.

The terms “half molecule”, “Fab-arm”, and “arm”, as used herein, refer to one heavy chain-light chain pair.

When a bispecific antibody is described as comprising a half-molecule antibody “derived from” a first parental antibody, and a half-molecule antibody “derived from” a second parental antibody, the term “derived from” indicates that the bispecific antibody was generated by recombining, by any known method, said half-molecules from each of said first and second parental antibodies into the resulting bispecific antibody. In this context, “recombining” is not intended to be limited by any particular method of recombining and thus includes all of the methods for producing bispecific antibodies described herein, including for example recombining by half-molecule exchange (also known as “controlled Fab-arm exchange”), as well as recombining at nucleic acid level and/or through co-expression of two half-molecules in the same cells.

The term “full-length” as used herein in the context of an antibody indicates that the antibody is not a fragment but contains all of the domains of the particular isotype normally found for that isotype in nature, e.g., the VH, CH1, CH2, CH3, hinge, VL and CL domains for an IgG1 antibody. A full-length antibody may be engineered. An example of a “full-length” antibody is epcoritamab.

The term “Fc region” as used herein refers to an antibody region consisting of the Fc sequences of the two heavy chains of an immunoglobulin, wherein said Fc sequences comprise at least a hinge region, a CH2 domain, and a CH3 domain.

The term “heterodimeric interaction between the first and second CH3 regions” as used herein refers to the interaction between the first CH3 region and the second CH3 region in a first-CH3/second-CH3 heterodimeric protein.

The term “homodimeric interactions of the first and second CH3 regions” as used herein refers to the interaction between a first CH3 region and another first CH3 region in a first-CH3/first-CH3 homodimeric protein and the interaction between a second CH3 region and another second CH3 region in a second-CH3/second-CH3 homodimeric protein.

The term “binding” as used herein in the context of the binding of an antibody to a predetermined antigen typically refers to binding with an affinity corresponding to a K_{D} of about 10⁻⁶ M or less, e.g., 10⁻⁷ M or less, such as about 10⁻⁸ M or less, such as about 10⁻⁹ M or less, about 10⁻¹⁰ M or less, or about 10⁻¹¹ M or even less, when determined by, e.g., BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (i.e., the antibody is highly specific).

The term “K_{D}” (M) as used herein refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “isolated antibody” as used herein refers to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95LI7), a CD3δ (delta) chain (human CD36 UniProtKB/Swiss-Prot No P04234, or cynomolgus monkey CD36 UniProtKB/Swiss-Prot No Q95LI8), two CD3ε(epsilon) chains (human CD3ε UniProtKB/Swiss-Prot No P07766, SEQ ID NO: 28); cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ζ UniProtKB/Swiss-Prot No P20963, cynomolgus monkey CD3ζ (UniProtKB/Swiss-Prot No Q09TK0). These chains associate with a molecule known as the T-cell receptor (TCR) and generate an activation signal in T lymphocytes. The TCR and CD3 molecules together comprise the TCR complex.

The term “CD3 antibody” or “anti-CD3 antibody” as used herein refers to an antibody which binds specifically to the antigen CD3, in particular human CD3ε (epsilon).

The term “human CD20” or “CD20” refers to human CD20 (UniProtKB/Swiss-Prot No P11836, SEQ ID NO: 29) and includes any variants, isoforms, and species homologs of CD20 which are naturally expressed by cells, including tumor cells, or are expressed on cells transfected with the CD20 gene or cDNA. Species homologs include rhesus monkey CD20 (macaca mulatta; UniProtKB/Swiss-Prot No H9YXP1) and cynomolgus monkey CD20 (Macaca fascicularis; UniProtKB No G7PQ03).

The term “CD20 antibody” or “anti-CD20 antibody” as used herein refers to an antibody which binds specifically to the antigen CD20, in particular to human CD20.

The term “CD3xCD20 antibody”, “anti-CD3xCD20 antibody”, “CD20xCD3 antibody” or “anti-CD20xCD3 antibody” as used herein refers to a bispecific antibody which comprises two different antigen-binding regions, one of which binds specifically to the antigen CD20 and one of which binds specifically to CD3.

The term “DuoBody-CD3xCD20” as used herein refers to an IgG1 bispecific CD3xCD20 antibody comprising a first heavy and light chain pair as defined in SEQ ID NO: 24 and SEQ ID NO: 25, respectively, and comprising a second heavy and light chain pair as defined in SEQ ID NO: 26 and SEQ ID NO: 27. The first heavy and light chain pair comprises a region which binds to human CD3ε (epsilon), the second heavy and light chain pair comprises a region which binds to human CD20. The first binding region comprises the VH and VL sequences as defined by SEQ ID NOs: 6 and 7, and the second binding region comprises the VH and VL sequences as defined by SEQ ID NOs: 13 and 14. This bispecific antibody can be prepared as described in WO 2016/110576.

Antibodies comprising functional variants of the heavy chain, light chains, VL regions, VH regions, or one or more CDRs of the antibodies of the examples as also provided herein. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity for particular epitopes of CD20 and/or CD3, Fc inertness and PK parameters such as half-life, Tmax, Cmax. Such functional variants typically retain significant sequence identity to the parent antibody and/or have substantially similar length of heavy and light chains. The percent identity between two sequences is a function of the number of identical positions shared by the sequences (i.e., % homology=# of identical positions/total # of positions ×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm. Exemplary variants include those which differ from heavy and/or light chains, VH and/or VL, and/or CDR regions of the parent antibody sequences mainly by conservative substitutions; e.g., 10, such as 9, 8, 7, 6, 5, 4, 3, 2 or 1 of the substitutions in the variant may be conservative amino acid residue replacements.

Conservative substitutions may be defined by substitutions within the classes of amino acids reflected in the following table:

TABLE 1
Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Unless otherwise indicated, the following nomenclature is used to describe a mutation: i) substitution of an amino acid in a given position is written as, e.g., K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

The term “humanized antibody” as used herein refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. The VH and VL of the CD3 arm that is used herein in DuoBody-CD3xCD20 represents a humanized antigen-binding region. Optionally, additional amino acid modifications, which are not necessarily back-mutations, may be applied to obtain a humanized antibody with preferred characteristics, such as affinity and biochemical properties.

The term “human antibody” as used herein refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The VH and VL of the CD20 arm that is used in DuoBody-CD3xCD20 represents a human antigen-binding region. Human monoclonal antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus be generated using, e.g., transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery (see, e.g., Mendez et al. Nat Genet 1997; 15:146-56). The VH and VL regions of the CD20 arm that is used in DuoBody-CD3xCD20 represents a human antigen-binding region.

The term “biosimilar” (e.g., of an approved reference product/biological drug) as used herein refers to a biologic product that is similar to the reference product based on data from (a) analytical studies demonstrating that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product). In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. A biosimilar can be, e.g., a presently known antibody having the same primary amino acid sequence as a marketed antibody, but may be made in different cell types or by different production, purification, or formulation methods.

The term “reducing conditions” or “reducing environment” as used herein refers to a condition or an environment in which a substrate, here a cysteine residue in the hinge region of an antibody, is more likely to become reduced than oxidized.

The term “recombinant host cell” (or simply “host cell”) as used herein is intended to refer to a cell into which an expression vector has been introduced, e.g., an expression vector encoding an antibody described herein. Recombinant host cells include, for example, transfectomas, such as CHO, CHO-S, HEK, HEK293, HEK-293F, Expi293F, PER.C6 or NS0 cells, and lymphocytic cells.

As used herein, “Richter's syndrome” or “Richter's transformation” are used interchangeably to refer to the transformation of chronic lymphocytic leukemia (CLL) into an aggressive lymphoma. Richter's syndrome arises in the background of CLL or SLL (Swerdlow et al., 2017; WHO Classification of Tumours of Haematopoietic and Lymphoid Tissues. International Agency for Research on Cancer, Lyon, France) and occurs in approximately 10% to 15% of patients with CLL. In the majority of cases, CLL evolves into a diffuse large B-cell lymphoma (DLBCL) that maintains a clonal relationship with the original leukemic phase, whereas the rest of the patients develop a Hodgkin lymphoma variant.2 Survival of patients with RS is generally poor, and subjects carrying selective chromosomal aberrations or being clonally related to CLL experience the worst prognosis and outcome (Allan and Furman, 2019; Int J Hematol Oncol, 7 (4), p. IJHO9, Falchi, et al., 2014; Blood, 123 (18), pp. 2783-27903).

Several genetic and immune factors may contribute to the transformation. In recent years, additional risk factors have been identified, such as TP53 disruption, NOTCH1 mutation, CDKN2A loss, and MYC activation (Rossi, et al., 2018; Blood, 131 (25), pp. 2761-2772, Chigrinova, et al., 2013; Blood, 122 (15), pp. 2673-2682, Fabbri, et al., 2013; J Exp Med, 210 (11) 13), pp. 2273-2288, Parikh et al., 2014; Blood, 123 (11), pp. 1647-1657). In addition, biased usage of subset 8 V4-39 stereotyped immunoglobulin gene increases the risk of RS development by 24-fold (Parikh, et al., 2013; Br J Haematol, 162 (6), pp. 774-782, Rossi, et al., 2009, Clin Cancer Res, 15 (13), pp. 4415-4422), suggesting a driving role of B-cell receptor (BCR) signaling in transformation. Overall, the molecular profile of RS is heterogeneous, lacking a unifying lesion, and does not overlap with the genetics of de novo DLBCL (Fabbri, et al., 2013; J Exp Med, 210 (11) 13), pp. 2273-2288). Deregulation of the underlying transcriptional programs and signaling pathways may account for the aggressive clinical phenotype of RS(Allan and Furman, 2019; Int J Hematol Oncol, 7 (4), p. IJH09).

The term “treatment” refers to the administration of an effective amount of a therapeutically active antibody described herein for the purpose of easing, ameliorating, arresting or eradicating (curing) symptoms or disease states such as CLL. Treatment may result in a complete response (CR), partial response (PR), or stable disease (SD), for example, as defined by Lugano criteria (Cheson et al., 2014) as shown in Table 2.

Treatment may be continued, for example, until disease progression (PD) or unacceptable toxicity.

The term “administering” or “administration” as used herein refers to the physical introduction of a composition (or formulation) comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a therapeutic agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. In the methods described herein, the bispecific antibody (e.g., epcoritamab) is administered subcutaneously. Other agents used in combination with the bispecific antibody, such as for cytokine release syndrome prophylaxis or tumor lysis syndrome (TLS) prophylaxis, may be administered via other routes, such as intravenously or orally.

The term “effective amount” or “therapeutically effective amount” refers to an amount effective, at dosages and for periods of time necessary, to achieve a desired therapeutic result. For example, dosages as defined herein for the bispecific antibody (e.g., epcoritamab) in the range of 12-60 mg administered subcutaneously can be defined as such an “effective amount” or “therapeutically effective amount”. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects. In some embodiments, patients treated with the methods described herein will show an improvement in ECOG performance status. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or disorder (e.g., cytokine release syndrome) or of suffering a recurrence of disease, inhibits the development or recurrence of the disease.

The term “inhibits growth” of a tumor as used herein includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The term “subject” as used herein refers to a human patient, for example, a human patient with Richter's Syndrome. The terms “subject” and “patient” are used interchangeably herein.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

“Disease progression” or “PD” as used herein refers to a situation in which one or more indices of lymphoma show that the disease is advancing despite treatment. In some embodiments, disease progression is defined based on Lugano criteria (Cheson et al., 2014) as shown in Table 2.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/v or less, such as 3% w/v or less, such as 1% w/v or less such as 0.4% w/v or less, such as below 0.1% w/v or less, such as 0.04% w/v). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates such as polysorbate 20 or polysorbate 80, propylene glycol dilaurate, propylene glycol monolaurate, sorbitan esters sucrose palmitate, sucrose stearate, tricaprylin and TPGS (Nonionic and zwitterionic surfactants).

A “diluent” as used herein is one which is pharmaceutically acceptable (safe and non-toxic for administration to a human) and is useful for the preparation of dilutions of the pharmaceutical composition or pharmaceutical formulation (the terms “composition” and “formulation” are used interchangeably herein). Preferably, such dilutions of the composition dilute only the antibody concentration but not the buffer and stabilizer. Accordingly, in one embodiment, the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution, Ringer's solution or dextrose solution. In one embodiment the diluent comprises or consists essentially of acetate buffer and sorbitol.

As used herein, the term “about” refers to ±10% of a specified value.

### Richter's Syndrome Treatment Regimens

Richter's Syndrome (RS), also known as Richter's Transformation, is a rare complication of Chronic Lymphocytic Leukemia (CLL) and/or Small Lymphocytic Lymphoma (SLL). It is characterised by the sudden transformation of the CLL/SLL into a significantly more aggressive form of large cell lymphoma. In the most cases the normally slow growing, or indolent, CLL transforms into a common type of non-Hodgkin lymphoma (NHL) known as Diffuse Large B-Cell Lymphoma (DLBCL). Rarer cases transform into Hodgkin lymphoma (HL)/Hodgkin Disease (HD), and some types of T-cell lymphomas also have been reported.

While the exact causes of Richter's Syndrome remain unclear, certain factors are thought to increase the risk of developing RS in patients already diagnosed with CLL/SLL. These risk factors include certain inherited genetic characteristics (e.g., BCL-2, CD38, LRP4 genotypes), and specific genetic mutations. For example, patients harboring 11q and 17p chromosome deletions, un-mutated IGVH gene, NOTCH−1 mutations, shortened telomere length, elevated zeta associated protein (ZAP-70) beta 2 microglobulin (B2M) and CD38 levels and/or with advanced stage disease at first CLL diagnosis (Rai Stage III-IV with lymph nodes>3 cm) are all thought to be at a greater risk of developing RS.

Richter's syndrome is characterized by sudden clinical deterioration. Currently available therapies have shown limited responses, with unsatisfactory safety profiles. Median overall survival ranges from a few months to approximately 1 year. As there is no established standard of care, there is a need to provide therapies with novel modes of action that provide durable responses with a tolerable safety profile. One such therapy is to treat RS patients with a bispecific antibody which binds to CD3 and CD20 (“anti-CD3xCD20 antibody”).

Accordingly, in one aspect, provided herein is a method of treating Richter's syndrome in a human subject, the method comprising administering (e.g., subcutaneously) to the subject an effective amount of a bispecific antibody comprising:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14.

Preferably, the bispecific antibody is administered at a dose ranging from 12-60 mg in 28-days cycles.

In some embodiments, the bispecific antibody is a full-length antibody. In some embodiments, the bispecific antibody is an antibody with an inert Fc region. In some embodiments, the bispecific antibody is a full-length antibody with an inert Fc region.

In some embodiments, the bispecific antibody is administered at a dose of (or a dose of about) 12 mg. In some embodiments, the bispecific antibody is administered at a dose of (or a dose of about) 24 mg. In some embodiments, the bispecific antibody is administered at a dose of (or a dose of about) 48 mg. In some embodiments, the bispecific antibody is administered at a dose of (or a dose of about) 60 mg.

With regard to the dose of 12-60 mg of the bispecific antibody that is to be administered, or any other specified dose, it is understood that this amount refers to the amount of a bispecific antibody representing a full-length antibody, such as epcoritamab as defined in the Examples section. Hence, one may refer to administering a dose of a bispecific antibody of 24 mg as administering a dose of a bispecific antibody described herein, wherein the dose corresponds to a dose of 24 mg of epcoritamab. One of ordinary skill in the art can readily determine the amount of antibody to be administered when, for example, the antibody used differs substantially in molecular weight from the molecular weight of a full-length antibody such as epcoritamab. For instance, the amount of antibody can be calculated by dividing the molecular weight of the antibody by the weight of a full-length antibody such as epcoritamab and multiplying the outcome thereof with the specified dose as described herein. As long as the bispecific antibody (e.g., a functional variant of DuoBody-CD3xCD20) has highly similar features as DuoBody-CD3xCD20, with regard to plasma half-life, Fc inertness, and/or binding characteristics for CD3 and CD20, i.e., with regard to CDRs and epitope binding features, such antibodies are suitable for use in the methods provided herein at a dose described for a full-length antibody such as epcoritamab.

In one embodiment, the bispecific anti-CD3xCD20 antibody is administered at a dose in the range of between 12 mg and 60 mg. In some embodiments, the bispecific antibody is administered at a dose of 12 mg or about 12 mg. In some embodiments, the bispecific antibody is administered at a dose of 24 mg or about 24 mg. In some embodiments, the bispecific antibody is administered at a dose of 48 mg or about 48 mg. In some embodiments, the bispecific antibody is administered at a dose of 60 mg or about 60 mg.

In some embodiments, the dose of bispecific antibody is administered once every week (weekly administration) in 28-day cycles. In some embodiments, the weekly administration is performed for 2.5 28-day cycles (i.e., 10 times). In one embodiment, the dose is administered for 2.5 28-day cycles (i.e., 10 times; on days 15 and 22 of cycle 1, and days 1, 8, 15, and 22 of cycles 2 and 3). In some embodiments, after said weekly administration, one may reduce the interval of administrating the bispecific antibody to an administration once every two weeks (biweekly administration). In some embodiments, such biweekly administration may be performed for six 28-day cycles (i.e., 12 times). In some embodiments, after said biweekly administration, the interval of administrating the bispecific antibody may be reduced further to once every four weeks. In one embodiment, the administration once every four weeks may be performed for an extended period, for example, for at least 1 cycle, at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, at least 15 cycles, at least 16 cycles, at least 17 cycles, or between 1-20 cycles, 1-19 cycles, 1-18 cycles, 1-17 cycles, 1-16 cycles, 1-15 cycles, 1-14 cycles, 1-13 cycles, 1-12 cycles, 1-10 cycles, 1-5 cycles, 5-20 cycles, 5-15 cycles, or 5-10 cycles of the 28-day cycles. In some embodiments, epcoritamab is administered once every four weeks until disease progression (e.g., as defined by the Lugano criteria (Cheson et al., 2014) as shown in Table 2.) or unacceptable toxicity. In one embodiment, the weekly dose is administered on cycles 1-3 (and may include priming and intermediate doses, as described below), the biweekly dose is administered on cycles 4-9, and the dose once every four weeks is administered from cycle 10 onward.

It is understood that the doses referred to herein may also be referred to as a full or a flat dose in the scenarios above wherein, e.g., the weekly dose, the biweekly dose, and/or the dose every four weeks is administered is at the same level. Accordingly, when a dose of 48 mg is selected, preferably, at each weekly administration, each biweekly administration, and each administration every four weeks, the same dose of 48 mg is administered. Prior to administering the dose, a priming or a priming and one or more subsequent intermediate (second priming) dose(s) may be administered. This may be advantageous as it may help mitigate cytokine release syndrome (CRS) risk and severity, a side-effect that can occur during treatment with the bispecific anti-CD3xCD20 antibody described herein. Such priming, or priming and intermediate doses, are at a lower dose as compared with the flat or full dose.

Accordingly, in some embodiments, prior to administering the weekly dose of 12-60 mg, a priming dose of the bispecific antibody may be administered. In one embodiment, the priming dose is administered two weeks prior to administering the first weekly dose of 12-60 mg in cycle 1. The priming dose may be in the range of 20-2000 μg (0.02 mg-2 mg), for example, in the range of 50-1000 μg (0.05 mg to 1 mg) or in the range of 70-350 μg (0.07 mg to 0.35 mg). The priming dose can be, for example, 80, 100, 120, 140, 160, 180, 200, 220, 240, 260, 280, 300, 320, 350, 400, 450, 500, 600, 700, 800, 900, or 1000 μg, or about 80, about 100, about 120, about 140, about 160, about 180, about 200, about 220, about 240, about 260, about 280, about 300, about 320, about 350, about 400, about 450, about 500, about 600, about 700, about 800, about 900, or about 1000 μg. In a preferred embodiment, the priming dose is in the range of 50 and 350 μg (0.05 and 0.35 mg, respectively). In a more preferred embodiment, the priming dose is 160 μg (0.16 mg) or about 160 μg (about 0.16 mg). In most preferred embodiments, the priming dose is 160 μg (0.16 mg) or about 160 μg (about 0.16 mg) of the full-length bispecific antibody.

In some embodiments, after administering the priming dose and prior to administering the first weekly dose of 12-60 mg, one or more intermediate doses of said bispecific antibody is/are administered. In one embodiment, the priming dose is administered on day 1 and an intermediate dose is administered on day 8 before the first weekly dose of 12-60 mg on days 15 and 22 of cycle 1 i.e. the priming dose is administered one week before the intermediate dose (i.e., day 1 of cycle 1), and an intermediate dose is administered one week before the first weekly dose of 12-60 mg (day 8 of cycle 1). The one or more intermediate doses is/are selected from a range in between the priming dose and the flat or full dose. For example, the one or more intermediate doses may be in the range of 200-8000 μg (0.2-8 mg), e.g., in the range of 400-4000 μg (0.4-4 mg) or 600-2000 μg (0.6-2 mg). An intermediate dose can be, for example, 200, 300, 400, 500, 600, 700, 800, 900, 1000, 1100, 1200, 1300, 1400, 1500, or 1600 μg, or about 200, about 300, about 400, about 500, about 600, about 700, about 800, about 900, about 1000, about 1100, about 1200, about 1300, about 1400, about 1500, or about 1600 μg. In a preferred embodiment, the intermediate dose is in the range of 600 and 1200 μg (0.6 and 1.2 mg, respectively). A currently preferred embodiment uses an intermediate dose, which is 800 μg (0.8 mg) or about 800 μg (0.8 mg). A most preferred embodiment uses an intermediate dose, which is 800 μg or about 800 μg (0.8 mg) of the full-length bispecific antibody.

In some embodiments, the bispecific antibody is administered (e.g., subcutaneously) in 28-day cycles, wherein
a) in cycle 1, a priming dose is administered on day 1, an intermediate dose on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is administered (e.g., subcutaneously) in 28-day cycles, wherein
a) in cycle 1, a priming dose of 160 μg is administered on day 1, an intermediate dose of 800 μg on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is administered (e.g., subcutaneously) in 28-day cycles, wherein
a) in cycle 1, a priming dose is administered on day 1, an intermediate dose on day 8, and a full dose of 12 mg or about 12 mg on days 15 and 22;
b) in cycles 2-3, a full dose of 12 mg or about 12 mg is administered on days 1, 8, 15, and 22;
c) in cycles 4-9, a full dose of 12 mg or about 12 mg is administered on days 1 and 15; and
d) in cycle 10 and subsequent cycles, a full dose of 12 mg or about 12 mg is administered on day 1.

In some embodiments, the bispecific antibody is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 12 mg on days 15 and 22;
b) in cycles 2-3, a full dose of 12 mg is administered on days 1, 8, 15, and 22;
c) in cycles 4-9, a full dose of 12 mg is administered on days 1 and 15; and
d) in cycle 10 and subsequent cycles, a full dose of 12 mg is administered on day 1.

In some embodiments, the bispecific antibody is administered (e.g., subcutaneously) in 28-day cycles, wherein
a) in cycle 1, a priming dose of 160 μg is administered on day 1, an intermediate dose of 800 μg on day 8, and a full dose of 12 mg or about 12 mg on days 15 and 22;
b) in cycles 2-3, a full dose of 12 mg or about 12 mg is administered on days 1, 8, 15, and 22;
c) in cycles 4-9, a full dose of 12 mg or about 12 mg is administered on days 1 and 15; and
d) in cycle 10 and subsequent cycles, a full dose of 12 mg or about 12 mg is administered on day 1.

In some embodiments, the bispecific antibody is administered (e.g., subcutaneously) in 28-day cycles, wherein
a) in cycle 1, a priming dose is administered on day 1, an intermediate dose on day 8, and a full dose of 24 mg or about 24 mg on days 15 and 22;
b) in cycles 2-3, a full dose of 24 mg or about 24 mg is administered on days 1, 8, 15, and 22;
c) in cycles 4-9, a full dose of 24 mg or about 24 mg is administered on days 1 and 15; and
d) in cycle 10 and subsequent cycles, a full dose of 24 mg or about 24 mg is administered on day 1.

In some embodiments, the bispecific antibody is administered (e.g., subcutaneously) in 28-day cycles, wherein
a) in cycle 1, a priming dose in the range of 0.05 and 0.35 is administered on day 1, an intermediate dose in the range of 0.6 and 1.2 mg on day 8, and a full dose of 24 mg on days 15 and 22;
b) in cycles 2-3, a full dose of 24 mg is administered on days 1, 8, 15, and 22;
c) in cycles 4-9, a full dose of 24 mg is administered on days 1 and 15; and
d) in cycle 10 and subsequent cycles, a full dose of 24 mg is administered on day 1.

In some embodiments, the bispecific antibody is administered (e.g., subcutaneously) in 28-day cycles, wherein
a) in cycle 1, a priming dose of 160 μg is administered on day 1, an intermediate dose of 800 μg on day 8, and a full dose of 24 mg or about 24 mg on days 15 and 22;
b) in cycles 2-3, a full dose of 24 mg or about 24 mg is administered on days 1, 8, 15, and 22;
c) in cycles 4-9, a full dose of 24 mg or about 24 mg is administered on days 1 and 15; and
d) in cycle 10 and subsequent cycles, a full dose of 24 mg or about 24 mg is administered on day 1.

In some embodiments, the bispecific antibody is administered (e.g., subcutaneously) in 28-day cycles, wherein
a) in cycle 1, a priming dose is administered on day 1, an intermediate dose on day 8, and a full dose of 48 mg or about 48 mg on days 15 and 22;
b) in cycles 2-3, a full dose of 48 mg or about 48 mg is administered on days 1, 8, 15, and 22;
c) in cycles 4-9, a full dose of 48 mg or about 48 mg is administered on days 1 and 15; and
d) in cycle 10 and subsequent cycles, a full dose of 48 mg or about 48 mg is administered on day 1.

In some embodiments, the bispecific antibody is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 48 mg on days 15 and 22;
b) in cycles 2-3, a full dose of 48 mg is administered on days 1, 8, 15, and 22;
c) in cycles 4-9, a full dose of 48 mg is administered on days 1 and 15; and
d) in cycle 10 and subsequent cycles, a full dose of 48 mg is administered on day 1.

In some embodiments, the bispecific antibody is administered (e.g., subcutaneously) in 28-day cycles, wherein
a) in cycle 1, a priming dose of 160 μg is administered on day 1, an intermediate dose of 800 μg on day 8, and a full dose of 48 mg or about 48 mg on days 15 and 22;
b) in cycles 2-3, a full dose of 48 mg or about 48 mg is administered on days 1, 8, 15, and 22;
c) in cycles 4-9, a full dose of 48 mg or about 48 mg is administered on days 1 and 15; and
d) in cycle 10 and subsequent cycles, a full dose of 48 mg or about 48 mg is administered on day 1.

In some embodiments, the bispecific antibody is administered (e.g., subcutaneously) in 28-day cycles, wherein
a) in cycle 1, a priming dose is administered on day 1, an intermediate dose on day 8, and a full dose of 60 mg or about 60 mg on days 15 and 22;
b) in cycles 2-3, a full dose of 60 mg or about 60 mg is administered on days 1, 8, 15, and 22;
c) in cycles 4-9, a full dose of 60 mg or about 60 mg is administered on days 1 and 15; and
d) in cycle 10 and subsequent cycles, a full dose of 60 mg or about 60 mg is administered on day 1.

In some embodiments, the bispecific antibody is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 60 mg on days 15 and 22;
b) in cycles 2-3, a full dose of 60 mg is administered on days 1, 8, 15, and 22;
c) in cycles 4-9, a full dose of 60 mg is administered on days 1 and 15; and
d) in cycle 10 and subsequent cycles, a full dose of 60 mg is administered on day 1.

In some embodiments, the bispecific antibody is administered (e.g., subcutaneously) in 28-day cycles, wherein
a) in cycle 1, a priming dose of 160 μg is administered on day 1, an intermediate dose of 800 μg on day 8, and a full dose of 60 mg or about 60 mg on days 15 and 22;
b) in cycles 2-3, a full dose of 60 mg or about 60 mg is administered on days 1, 8, 15, and 22;
c) in cycles 4-9, a full dose of 60 mg or about 60 mg is administered on days 1 and 15; and
d) in cycle 10 and subsequent cycles, a full dose of 60 mg or about 60 mg is administered on day 1.

In some embodiments, the bispecific antibody is epcoritamab, which is administered subcutaneously in 28-day cycles, wherein
a) in cycle 1, a priming dose is administered on day 1, an intermediate dose on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is epcoritamab, which is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is epcoritamab, which is administered subcutaneously in 28-day cycles, wherein
a) in cycle 1, a priming dose of 160 μg is administered on day 1, an intermediate dose of 800 μg on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

In some embodiments, the bispecific antibody is epcoritamab, which is administered subcutaneously in 28-day cycles, wherein
a) in cycle 1, a priming dose is administered on day 1, an intermediate dose on day 8, and a full dose of 12 mg or about 12 mg on days 15 and 22;
b) in cycles 2-3, a full dose of 12 mg or about 12 mg is administered on days 1, 8, 15, and 22;
c) in cycles 4-9, a full dose of 12 mg or about 12 mg is administered on days 1 and 15; and
d) in cycle 10 and subsequent cycles, a full dose of 12 mg or about 12 mg is administered on day 1.

In some embodiments, the bispecific antibody is epcoritamab, which is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 12 mg on days 15 and 22;
b) in cycles 2-3, a full dose of 12 mg is administered on days 1, 8, 15, and 22;
c) in cycles 4-9, a full dose of 12 mg is administered on days 1 and 15; and
d) in cycle 10 and subsequent cycles, a full dose of 12 mg is administered on day 1.

In some embodiments, the bispecific antibody is epcoritamab, which is administered subcutaneously in 28-day cycles, wherein
a) in cycle 1, a priming dose of 160 μg is administered on day 1, an intermediate dose of 800 μg on day 8, and a full dose of 12 mg or about 12 mg on days 15 and 22;
b) in cycles 2-3, a full dose of 12 mg or about 12 mg is administered on days 1, 8, 15, and 22;
c) in cycles 4-9, a full dose of 12 mg or about 12 mg is administered on days 1 and 15; and
d) in cycle 10 and subsequent cycles, a full dose of 12 mg or about 12 mg is administered on day 1.

In some embodiments, the bispecific antibody is epcoritamab, which is administered subcutaneously in 28-day cycles, wherein
a) in cycle 1, a priming dose is administered on day 1, an intermediate dose on day 8, and a full dose of 24 mg or about 24 mg on days 15 and 22;
b) in cycles 2-3, a full dose of 24 mg or about 24 mg is administered on days 1, 8, 15, and 22;
c) in cycles 4-9, a full dose of 24 mg or about 24 mg is administered on days 1 and 15; and
d) in cycle 10 and subsequent cycles, a full dose of 24 mg or about 24 mg is administered on day 1.

In some embodiments, the bispecific antibody is epcoritamab, which is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 24 mg on days 15 and 22;
b) in cycles 2-3, a full dose of 24 mg is administered on days 1, 8, 15, and 22;
c) in cycles 4-9, a full dose of 24 mg is administered on days 1 and 15; and
d) in cycle 10 and subsequent cycles, a full dose of 24 mg is administered on day 1.

In some embodiments, the bispecific antibody is epcoritamab, which is administered subcutaneously in 28-day cycles, wherein
a) in cycle 1, a priming dose of 160 μg is administered on day 1, an intermediate dose of 800 μg on day 8, and a full dose of 24 mg or about 24 mg on days 15 and 22;
b) in cycles 2-3, a full dose of 24 mg or about 24 mg is administered on days 1, 8, 15, and 22;
c) in cycles 4-9, a full dose of 24 mg or about 24 mg is administered on days 1 and 15; and
d) in cycle 10 and subsequent cycles, a full dose of 24 mg or about 24 mg is administered on day 1.

In some embodiments, the bispecific antibody is epcoritamab, which is administered subcutaneously in 28-day cycles, wherein
a) in cycle 1, a priming dose is administered on day 1, an intermediate dose on day 8, and a full dose of 48 mg or about 48 mg on days 15 and 22;
b) in cycles 2-3, a full dose of 48 mg or about 48 mg is administered on days 1, 8, 15, and 22;
c) in cycles 4-9, a full dose of 48 mg or about 48 mg is administered on days 1 and 15; and
d) in cycle 10 and subsequent cycles, a full dose of 48 mg or about 48 mg is administered on day 1.

In some embodiments, the bispecific antibody is epcoritamab, which is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 48 mg on days 15 and 22;
b) in cycles 2-3, a full dose of 48 mg is administered on days 1, 8, 15, and 22;
c) in cycles 4-9, a full dose of 48 mg is administered on days 1 and 15; and
d) in cycle 10 and subsequent cycles, a full dose of 48 mg is administered on day 1.

In some embodiments, the bispecific antibody is epcoritamab, which is administered subcutaneously in 28-day cycles, wherein
a) in cycle 1, a priming dose of 160 μg is administered on day 1, an intermediate dose of 800 μg on day 8, and a full dose of 48 mg or about 48 mg on days 15 and 22;
b) in cycles 2-3, a full dose of 48 mg or about 48 mg is administered on days 1, 8, 15, and 22;
c) in cycles 4-9, a full dose of 48 mg or about 48 mg is administered on days 1 and 15; and
d) in cycle 10 and subsequent cycles, a full dose of 48 mg or about 48 mg is administered on day 1.

In some embodiments, the bispecific antibody is epcoritamab, which is administered subcutaneously in 28-day cycles, wherein
a) in cycle 1, a priming dose is administered on day 1, an intermediate dose on day 8, and a full dose of 60 mg or about 60 mg on days 15 and 22;
b) in cycles 2-3, a full dose of 60 mg or about 60 mg is administered on days 1, 8, 15, and 22;
c) in cycles 4-9, a full dose of 60 mg or about 60 mg is administered on days 1 and 15; and
d) in cycle 10 and subsequent cycles, a full dose of 60 mg or about 60 mg is administered on day 1.

In some embodiments, the bispecific antibody is epcoritamab, which is administered (e.g., subcutaneously) in 28-day cycles, wherein
a) in cycle 1, a priming dose in the range of 0.05-0.35 is administered on day 1, an intermediate dose in the range of 0.6-1.2 mg on day 8, and a full dose of 60 mg on days 15 and 22;
b) in cycles 2-3, a full dose of 60 mg is administered on days 1, 8, 15, and 22;
c) in cycles 4-9, a full dose of 60 mg is administered on days 1 and 15; and
d) in cycle 10 and subsequent cycles, a full dose of 60 mg is administered on day 1.

In some embodiments, the bispecific antibody is epcoritamab, which is administered subcutaneously in 28-day cycles, wherein
a) in cycle 1, a priming dose of 160 μg is administered on day 1, an intermediate dose of 800 μg on day 8, and a full dose of 60 mg or about 60 mg on days 15 and 22;
b) in cycles 2-3, a full dose of 60 mg or about 60 mg is administered on days 1, 8, 15, and 22;
c) in cycles 4-9, a full dose of 60 mg or about 60 mg is administered on days 1 and 15; and
d) in cycle 10 and subsequent cycles, a full dose of 60 mg or about 60 mg is administered on day 1.

In one embodiment, on days 1 and 8 of the first cycle, a priming dose of 80 μg and an intermediate dose of 800 μg, respectively, is selected. In some embodiments, on days 1 and 8 of the first cycle, a priming dose of 80 μg and an intermediate dose of 1200 μg, respectively, is selected. In some embodiments, on days 1 and 8 of the first cycle, a priming dose of 80 μg and an intermediate dose of 1600 μg, respectively, is selected. In some embodiments, on days 1 and 8 of the first cycle, a priming dose of 160 μg and an intermediate dose of 1200 μg, respectively, is selected. In some embodiments, on days 1 and 8 of the first cycle, a priming dose of 160 μg and an intermediate dose of 1600 μg, respectively, is selected.

In one embodiment, the subject has clinical history of CLL/SLL with transformation toward aggressive lymphoma; e.g. of the DLBCL subtype. In a further embodiment, the Richter's syndrome is of the DLBCL subtype.

In one embodiment, the human subject has measurable disease as determined by both: a) fluorodeoxyglucose (FDG)-positron emission tomography (PET) CT scan demonstrating positive lesion compatible with CT (or MRI)-defined anatomical tumor sites; and b) A CT scan (or MRI) with involvement of ≥2 clearly demarcated lesions/nodes with long axis>1.5 cm and short axis>1.0 cm or 1 clearly demarcated lesion/node with a long axis>2.0 cm and a short axis≥1.0 cm.

In some embodiments, the human subject has received at least one line of treatment prior to being treated with the methods described herein. For instance, in one embodiment, the subject has received one prior line of treatment. In some embodiments, the subject has refractory and/or relapsed Richter's Syndrome after receiving one prior antineoplastic therapy. Relapse may be defined as evidence of disease progression in a subject who has previously achieved a CR or PR for at least 6 months. Refractory disease may be defined as treatment failure (not achieving a CR or PR) or as progression within 6 months from the last dose of therapy. In some embodiments, the subject has received three prior lines of treatment. In some embodiments, the subject has received more than three prior lines of treatment. In some embodiments, the subject has received one, two, three, or more prior lines of treatment. In some embodiments, the subject has received at least two prior lines of treatment. In one embodiment, a prior line of treatment comprises systemic antineoplastic therapy.

In some embodiments, the subject has received one or more, such as at least two, prior lines of therapy for Chronic Lymphocytic Leukemia (CLL) and/or for Small Lymphocytic Lymphoma (SLL).

The prior lines of therapy for CLL and/or SLL may in particular comprise chemoimmunotherapy.

In other embodiments, the prior lines of therapy for CLL and/or SLL comprise therapy with a targeted agent, such as BCL2 inhibitor or a BTK inhibitor.

In still further embodiments, the prior lines of therapy for CLL and/or SLL comprise CAR T-cell therapy.

In some embodiments, the subject has received prior therapy for Richter's syndrome, such as prior therapy selected from:
i) Rituximab in combination with cyclophosphamide, doxorubicin, vincristine, and prednisone (R-CHOP),
ii) Rituximab in combination with dexamethasone, cytarabine, and cisplatin (R-DHAP),
iii) Venetoclax in combination with rituximab, etoposide, prednisone, vincristine, cyclophosphamide, and doxorubicin (VR-EPOCH).

In some embodiments, the subject treated according to the invention achieves a complete metabolic response or a partial metabolic response.

In other embodiments, the subject treated according to the invention achieves a complete response, a partial response, or stable disease.

In other embodiments, the subject receives epcoritamab according to the invention as first-line therapy for Richter's syndrome. In further embodiments, the method according to the present invention is first-line therapy for Richter's syndrome.

According to these embodiments, the subject treated according to the invention as first-line therapy for Richter's syndrome achieves a complete metabolic response or a partial metabolic response.

In other embodiments, wherein the subject is treated according to the invention as first-line therapy for Richter's syndrome, the subject achieves a complete response, a partial response, or stable disease.

In some embodiments, the human subject must have a clinical history of CLL/SLL with biopsy-proven transformation toward aggressive lymphoma (ie, DLBCL subtype). In some embodiments, the human subject is deemed as ineligible for chemoimmunotherapy at investigator's discretion or refuse to receive intensive chemotherapy.

In some embodiments, the human subject must have measurable disease as determined by both: a) fluorodeoxyglucose (FDG)-positron emission tomography (PET) CT scan demonstrating positive lesion compatible with CT (or MRI)-defined anatomical tumor sites; and b) A CT scan (or MRI) with involvement of ≥2 clearly demarcated lesions/nodes with long axis>1.5 cm and short axis>1.0 cm or 1 clearly demarcated lesion/node with a long axis>2.0 cm and a short axis≥1.0 cm.

In some embodiments, the human subject has an ECOG performance status score of 0 or 2. Information regarding ECOG performance status scores can be found in, e.g., Oken et al, Am J Clin Oncol 1982 Dec. ;5(6):649-55).

In some embodiments, the human subject has acceptable laboratory parameters for (1) creatine clearance or serum creatine (>45 mL/min using Cockcroft-Gault formula or serum creatinine 1.5 times the upper limit of normal (x ULN)), (2) serum alanine transaminase (≤2.5×ULN), (3) serum aspartate transaminase (≤2.5×ULN), (≤4) bilirubin (1.5×ULN unless due to Gilbert syndrome), (5) hemoglobin (≥9.0 g/dL unless anemia is due to marrow involvement of CLL), (6) absolute neutrophil count (≥1.0×10⁹/L (1000/μL) unless neutropenia is due to bone marrow involvement of CLL), platelet count (≥30×10⁹/L (30,000/μL)), and coagulation status (PT/INR/aPTT≥1.5×ULN).

A human subject receiving a treatment described herein may be a patient having one or more of the inclusion criteria set forth in Example 2, or not having one or more of the exclusion criteria set forth in Example 2.

Human subjects with Richter's Syndrome are classified as having a CD20-positive cancer. Thus, prior cancer treatments such human subjects may have received include anti-CD20 monoclonal antibodies (e.g., rituximab). During such treatments, or any other treatments, the RS may be refractory or have relapsed to said treatment. Accordingly, in one embodiment, the subject has received prior to treatment with the bispecific antibody a treatment with an anti-CD20 monoclonal antibody, such as rituximab or obinutuzumab. In some embodiments, during said prior treatment with the anti-CD20 antibody or combinations of anti-CD20 monoclonal antibody with one therapeutic agent, e.g., cyclophosphamide, doxyrubicin hydrochloride, vincristine sulfate, prednisone (R-CHOP)), the RSrelapsed or was refractory to treatment.

The methods described herein are advantageous for treating Richter's syndrome. However, treatment may be terminated when progressive disease develops or unacceptable toxicity occurs.

The response of subjects with RS to the methods described herein may be assessed according to Lugano criteria (Cheson et al., 2014) as shown in Table 2.

TABLE 2
Lugano criteria (Cheson et al., 2014)

| Response | Site | PET-CT-based response | CT-based response |
|---|---|---|---|
| Complete | | Complete metabolic response | Complete radiologic |
| response | | | response |
| | Lymph nodes | Score 1, 2, or 3¹ with or | Target nodes/nodal |
| | and | without a residual mass on | masses must regress |
| | extralymphatic | 5PS2 It is recognized that in | to ≤1.5 cm in LDi. No |
| | sites | Waldeyer's ring or extranodal | extralymphatic sites of |
| | | sites with high physiologic | disease |
| | | uptake or with activation | |
| | | within spleen or marrow (eg, | |
| | | with chemotherapy or myeloid | |
| | | colony-stimulating factors), | |
| | | uptake may be greater than | |
| | | normal mediastinum and/or | |
| | | liver. In this circumstance, | |
| | | complete metabolic response | |
| | | may be inferred if uptake at | |
| | | sites of initial involvement is | |
| | | no greater than surrounding | |
| | | normal tissue even if the tissue | |
| | | has high physiologic uptake. | |
| | Non-measured | Non-applicable | Absent |
| | lesion | | |
| | Organ | Non-applicable | Regress to normal |
| | enlargement | | |
| | New lesion | None | None |
| | Bone marrow | No evidence of FDG-avid | Normal by morphology; |
| | | disease in marrow | if indeterminate, IHC |
| | | | negative |
| Partial | | Partial metabolic response | Partial remission (all of |
| response | | | the following) |
| | Lymph nodes | Score 4 or 5² with reduced | ≥50% decrease in SPD |
| | and | uptake compared with baseline | of up to 6 target |
| | extralymphatic | and residual mass(es) of any | measurable nodes and |
| | sites | size. At interim, these findings | extranodal sites |
| | | suggest responding disease. At | When a lesion is too |
| | | end of treatment, these | small to measure on CT, |
| | | findings indicate residual | assign 5 mm × 5 mm as |
| | | disease. | the default value When |
| | | | no longer visible, 0 × 0 |
| | | | mm. For a node >5 mm × |
| | | | 5 mm, but smaller than |
| | | | normal, use actual |
| | | | measurement for |
| | | | calculation |
| | Non-measured | Not applicable | Absent/normal, |
| | lesion | | regressed, but no |
| | | | increase |
| | Organ | Not applicable | Spleen must have |
| | enlargement | | regressed by >50% in |
| | | | length beyond normal |
| | New lesion | None | None |
| | Bone marrow | Residual uptake higher than | Not applicable |
| | | uptake in normal marrow but | |
| | | reduced compared with | |
| | | baseline (diffuse uptake | |
| | | compatible with reactive | |
| | | changes from chemotherapy | |
| | | allowed). If there are persistent | |
| | | focal changes in the marrow in | |
| | | the context of a nodal | |
| | | response, consideration should | |
| | | be given to further evaluation | |
| | | with MRI or biopsy or an | |
| | | interval scan. | |
| No response | | No metabolic response | Stable disease |
| or Stable | Target | Score 4 or 5² with no | <50% decrease from |
| disease | nodes/nodal | significant change in FDG | baseline in SPD of up to |
| | masses, | uptake from baseline at interim | 6 dominant, measurable |
| | extranodal | or end of treatment | nodes and extranodal |
| | lesions | | sites; no criteria for |
| | | | progressive disease are |
| | | | met |
| | Non-measured | Not applicable | No increase consistent |
| | lesion | | with progression |
| | Organ | Not applicable | No increase consistent |
| | enlargement | | with progression |
| | New lesion | None | None |
| | Bone marrow | No change from base line | Not applicable |
| Progressive | | Progressive metabolic disease | Progressive disease |
| disease | | | requires at least on the |
| | | | following |
| | Individual | Score 4 or 5² with an increase | PPD progression: An |
| | target | in intensity of uptake from | individual node/lesion |
| | nodes/nodal | baseline and/or New FDG- | must be abnormal with: |
| | masses, | avid foci consistent with | LDi >1.5 cm and |
| | extranodal | lymphoma at interim or end- | Increase by ≥50% from |
| | lesions | of-treatment assessment | PPD nadir and An |
| | | | increase in LDi or Sdi |
| | | | from nadir |
| | | | 0.5 cm for lesions ≤2 |
| | | | cm |
| | | | 1.0 cm for lesions ≤2 |
| | | | cm. |
| | | | In the setting of |
| | | | splenomegaly (>13 cm), |
| | | | the splenic length must |
| | | | increase by >50% of the |
| | | | extent of its prior |
| | | | increase beyond baseline |
| | | | (eg, a 15-cm spleen must |
| | | | increase to >16 cm). If |
| | | | no prior splenomegaly, |
| | | | must increase by at least |
| | | | 2 cm from baseline. New |
| | | | or recurrent splenomegaly |
| | Non-measured | None | esion |
| | lesion | | |
| | | | New or clear progression |
| | | | of pre-existing non- |
| | | | measured lesions |
| | New lesion | New FDG-avid foci consistent | Regrowth of previously |
| | | with lymphoma rather than | resolved lesions A new |
| | | another etiology (eg, infection, | node >1.5 cm in any axis |
| | | inflammation); if uncertain | A new extranodal site >1.0 |
| | | regarding etiology of new | cm in any axis; if <1.0 |
| | | lesions, biopsy or interval scan | cm in any axis, its |
| | | may be considered | presence must be |
| | | | unequivocal and must be |
| | | | attributable to lymphoma |
| | | | Assessable disease of any |
| | | | size unequivocally |
| | | | attributable to lymphoma |
| | Bone marrow | New or recurrent FDG-avid | New or recurrent |
| | | foci | involvement |

Abbreviations: 5PS = 5-point scale; CT = computed tomography; FDG = fluorodeoxyglucose; IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.

1. A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment).
Measured dominant (target) lesions: Up to six of the largest dominant nodes, nodal masses, and extranodal lesions selected to be clearly measurable in two diameters.
   Nodes should preferably be from disparate regions of the body and should include, where applicable, mediastinal and retroperitoneal areas.
   Non-nodal lesions include those in solid organs (eg, liver, spleen, kidneys, lungs), gastrointestinal involvement, cutaneous lesions, or those noted on palpation.
Non-measured lesions: Any disease not selected as measured, dominant disease and truly assessable disease should be considered not measured.
   These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging.
In Waldeyer's ring or in extranodal sites (eg, GI tract, liver, bone marrow), FDG uptake may be greater than in the mediastinum with complete metabolic response but should be no higher than surrounding normal physiologic uptake (eg, with marrow activation as a result of chemotherapy or myeloid growth factors).
   2. PET 5PS: 1=no uptake above background; 2=uptake≤mediastinum; 3=uptake>mediastinum but≤liver; 4=uptake moderately>liver; 5=uptake markedly higher than liver and/or new lesions; X=new areas of uptake unlikely to be related to lymphoma. Source: (Cheson et al., 2014, J Clin Oncol 32, 3059-3068) Subjects treated according to the methods described herein preferably experience improvement in at least one sign of lymphoma.

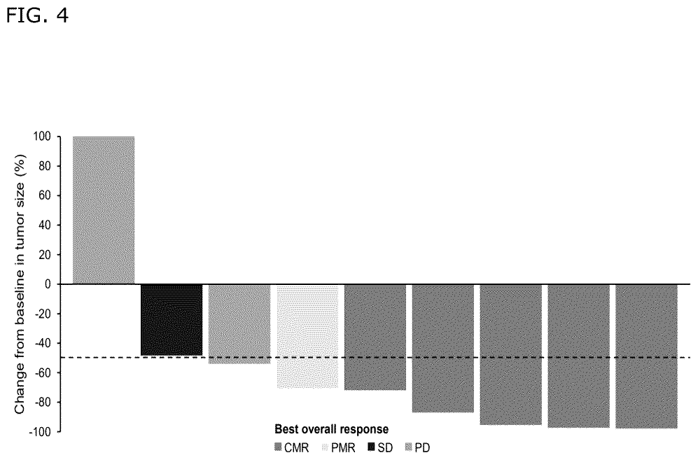

In one embodiment, the subject treated exhibits a complete metabolic response or a partial metabolic response as measured by PET (see Table 2, PET-CT based responses) and FIG. 4 showing that 8 out of 9 RS patients achieved at least 50% tumor reduction from baseline.

In one embodiment, the subject treated exhibits a complete response (CR), a partial response (PR), or stable disease (SD), as defined by Lugano criteria (Cheson et al., 2014) (see, e.g., Table 2). As shown in FIG. 4, 8 out 9 RS patients achieved at least a 50% reduction in tumor sized from baseline.

In some embodiments, the methods described herein produce at least one therapeutic effect chosen from prolonged survival, such as progression-free survival or overall survival, optionally compared to another therapy or placebo. In some embodiments, the subjects are treated with the methods described herein until disease progression (PD) or unacceptable toxicity.

Cytokine release syndrome (CRS) can occur when methods are used in human subjects that utilize immune cell- and bispecific antibody-based approaches that function by activation of immune effector cell, such as by engaging CD3 (Lee et al., Biol Blood Marrow Transplant 2019; 25:625-38, which is incorporated herein by reference). Hence, in some embodiments, CRS mitigation is performed together with the methods described herein. As part of CRS mitigation, the selection of a priming dose and/or intermediate dose is performed prior to administering the full dose (e.g., 12-60 mg), as described herein. CRS can be classified in accordance with standard practice (e.g. as outlined in Lee et al., Biol Blood Marrow Transplant. 2019 Apr.; 25(4):625-638, which is incorporated herein by reference). CRS may include excessive release of cytokines, for example of proinflammatory cytokines, e.g., IL-6, TNF-alpha or IL-8, that may result in adverse effects like fever, nausea, vomiting and chills. Thus, despite the unique anti-tumor activity of bispecific antibodies such as epcoritamab, their immunological mode of action may trigger unwanted “side” effects, i.e., the induction of unwanted inflammatory reactions. Hence, patients may be further subjected to a concomitant treatment, prophylaxis, and/or premedication with, e.g., analgesics, antipyretics, and/or anti-inflammatory drugs to mitigate possible CRS symptoms.

Accordingly, in one embodiment, human subjects in the methods described herein are treated with prophylaxis for CRS. In preferred embodiments, the prophylaxis comprises the administration of a corticosteroid to the subject. In one embodiment, the prophylaxis (e.g. corticosteroid) is administered on the same day as the bispecific antibody. The prophylaxis (e.g. corticosteroid) can also be administered on the subsequent days as well. In some embodiments, the prophylaxix (e.g. corticosteroid) is further administered on subsequent days 2, 3, and 4. It is understood that days 2, 3 and 4 when relating to further medication, such as prophylaxis, is relative to the administration of the bispecific antibody which is administered on day 1. For example, when in a cycle the antibody is administered on day 15, and prophylaxis is also administered, the prophylaxis corresponding to days 2, 3 and 4 are days 16, 17, and 18 of the cycle. In some embodiments, the prophylaxis is administered on the day when the bispecific antibody is administered and on subsequent days 2-4. When said prophylaxis is administered on the same day as the bispecific antibody, the prophylaxis is preferably administered 30-120 minutes prior to said administration of the bispecific antibody. An exemplary corticosteroid suitable for use in the methods and uses described herein is prednisolone. In some embodiments, the corticosteroid is prednisolone. In some embodiments, prednisolone is administered at an intravenous dose of 100 mg, or an equivalent thereof, including an oral dose. Exemplary corticosteroid equivalents of prednisolone, along with dosage equivalents, which can be used for CRS prophylaxis are shown in Table 6.

Furthermore, in some embodiments, human subjects in the methods described herein are treated with premedication to reduce reactions to injections. In one embodiment, the premedication includes the administration of antihistamines. In some embodiments, the premedication includes the administration of antipyretics. In a further embodiment, the premedication includes systemic administration of antihistamines and antipyretics.

An exemplary antihistamine suitable for use in premedication is diphenhydramine. In some embodiments, the antihistamine is diphenhydramine. In one embodiment, diphenhydramine is administered at an intravenous or oral dose 50 mg, or an equivalent thereof. An exemplary antipyretic suitable for use in premedication is acetaminophen. In some embodiments, the antipyretic is acetaminophen. In one embodiment, acetaminophen is administered at an oral dose of 560-1000 mg, such as 650-1000 mg, or equivalent thereof In some embodiments, the premedication is administered on the same day as the bispecific antibody. In some embodiments, the premedication is administered on the same day as the bispecific antibody prior to the injection with the bispecific antibody, e.g., 30-120 minutes prior to administration of the bispecific antibody.

Premedication and/or prophylaxis can be administered at least in the initial phase of the treatment. In some embodiments, premedication and/or prophylaxis is administered during the first four administrations of the bispecific antibody. For example, the premedication and/or prophylaxis can be administered as described herein, during the first 28 day cycle of the bispecific antibody administration. In some embodiments, the premedication is administered during cycle 1. In some embodiments, the prophylaxis is administered during cycle 1.

Usually, risk of reactions during the initial treatment subsides after a few administrations, e.g., after the first four administrations (first cycle). Hence, and when the human subject does not experience CRS, prophylaxis for CRS may be stopped. However, when the human subject experiences a CRS greater than grade 1, CRS prophylaxis may continue. Likewise, premedication may also optionally continue. CRS grading can be performed as described in Tables 7 and 8.

In a further embodiment, in the methods described herein, the prophylaxis is administered during the second 28-day cycle i.e. cycle 2, when the human subject experiences CRS greater than grade 1 after the fourth i.e. last administration of the bispecific antibody in cycle 1. Furthermore, the prophylaxis can be continued during a subsequent cycle, when in the last administration of the bispecific antibody of the previous cycle, the human subject experiences CRS greater than grade 1. Any premedication may be optionally administered during the second cycle. In some embodiments, the premedication is administered during cycle 2. Further premedication may be optionally administered during subsequent cycles as well. In some embodiments, the premedication is administered during subsequent cycles (after cycle 2).

In one embodiment, premedication and prophylaxis for CRS is administered, wherein the premedication comprises an antihistamine such as diphenhydramine (e.g., at an intravenous or oral dose 50 mg, or an equivalent thereof) and the prophylaxis comprises an antipyretic such as acetaminophen (e.g., at an oral dose of 650-1000 mg, or an equivalent thereof), and a corticosteroid such as prednisolone (e.g., at an intravenous dose of 100 mg, or an equivalent thereof). In some embodiments, the premedication and prophylaxis is administered 30-120 minutes prior to administration of the bispecific antibody. On subsequent days 2, 3, and optionally day 4, further prophylaxis is administered comprising the systemic administration of a corticosteroid such as prednisolone (e.g., at an intravenous dose of 100 mg, or an equivalent thereof). In some embodiments, the premedication and prophylaxis schedule preferably is administered during the first four administrations of the bispecific antibody, e.g., during the first 28-day cycle of bispecific antibody administration described herein. Furthermore, subsequent cycles, in case of, e.g., CRS greater than grade 1 occurring during the last administration of the prior cycle, can include the same administration schedule, wherein the premedication as part of the administration schedule is optional.

During the treatment of a human subject with RS using the doses and treatment regimens described herein, CRS can be well managed while at the same time effectively controlling and/or treating RS. As described in the Examples, subjects treated with the methods described herein may experience manageable CRS. In some cases, subjects receiving the treatment described herein may develop CRS of grade 1 as defined in accordance with standard practice. In other cases, subjects may develop manageable CRS of grade 2 as defined in accordance with standard practice. Hence, subjects receiving the treatments described herein may have manageable CRS of grade 1 or grade 2 during as defined in accordance with standard practice. In accordance with standard classification for CRS, a grade 1 CRS includes a fever to at least 38° C., no hypotension, no hypoxia, and a grade 2 CRS includes a fever to at least 38° C. plus hypotension, not requiring vasopressors and/or hypoxia requiring oxygen by low flow nasal cannula or blow by. Such manageable CRS can occur during cycle 1. Human subjects receiving the treatments described herein may also have CRS greater than grade 2 during the treatments as defined in accordance with standard practice. Hence, human subjects receiving the treatments described herein may also have CRS of grade 3 during said treatments as defined in accordance with standard practice. Such manageable CRS may further occur during cycle 1 and subsequent cycles.

Human subjects treated according to the methods described herein may also experience pyrexia, fatigue, and injection site reactions. They may also experience neurotoxicity, partial seizures, agraphia related to CRS, or confusional state related to CRS.

As mentioned above, subjects may develop CRS during treatment with the methods described herein, despite having received CRS prophylaxis. CRS grading criteria are described in Tables 7 and 8.

In one embodiment, subject is administered antibiotics if the subject develops Grade 1 CRS i.e. subjects who develop Grade 1 CRS are treated with antibiotics if they present with infection. In some embodiments, the antibiotics are continued until neutropenia, if present, resolves. In some embodiments, subjects with Grade 1 CRS who exhibit constitutional symptoms are treated with NSAIDs.

In one embodiment, subjects who develop Grade 2 CRS are treated with intravenous fluid boluses and/or supplemental oxygen. In some embodiments, subjects who develop Grade 2 CRS are treated with a vasopressor. In some embodiments, subjects with Grade 2 CRS with comorbidities are treated with tocilizumab (a humanized antibody against IL-6 receptor, commercially available as, e.g., ACTEMRA©) and/or steroids (e.g., dexamethasone or its equivalent of methylprednisolone). In a further embodiment, a subject who presents with concurrent ICANS is administered dexamethasone. In yet a further embodiment, if the subject does not show improvement in CRS symptoms within, e.g., 6 hours, or if the subject starts to deteriorate after initial improvement, then a second dose of tocilizumab is administered together with a dose of corticosteroids. In some embodiments, if the subject is refractory to tocilizumab after three administrations, then additional cytokine therapy, e.g., an anti-IL-6 antibody (e.g., siltuximab) or an IL-1R antagonist (e.g., anakinra) is administered to the subject.

In one embodiment, subjects who develop Grade 3 CRS are treated with vasopressor (e.g., norepinephrine) support and/or supplemental oxygen. In some embodiments, subjects with Grade 3 CRS are treated with tocilizumab, or tocilizumab in combination with steroids (e.g., dexamethasone or its equivalent of methylprednisolone). In some embodiments, a subject who presents with concurrent ICANS is administered dexamethasone. In a further embodiment, if the subject is refractory to tocilizumab after three administrations, then additional cytokine therapy, e.g., an anti-IL-6 antibody (e.g., siltuximab) or an IL-1R antagonist (e.g., anakinra) is administered to the subject.

In one embodiment, subjects who develop Grade 4 CRS are treated with vasopressor support and/or supplemental oxygen (e.g., via positive pressure ventilation, such as CPAP, BiPAP, intubation, or mechanical ventilation). In some embodiments, the subject is administered at least two vasopressors if the subject develops Grade 4 CRS. In some embodiments, the subject is further administered a steroid i.e. the subject is administered tocilizumab and a steroid. In some embodiments, the steroid is dexamethasone. In some embodiments, the steroid is methylprednisolone. In a further embodiment, a subject who presents with concurrent ICANS is administered dexamethasone. In a further embodiment, if the subject is refractory to tocilizumab after three administrations, then additional cytokine therapy, e.g., an anti-IL-6 antibody (e.g., siltuximab) or an IL-1R antagonist (e.g., anakinra) is administered to the subject. In some embodiments, administration of tocilizumab is switched to administration of an anti-IL-6 antibody (e.g., siltuximab) if the subject is refractory to tocilizumab. In some embodiments, tocilizumab is switched to an IL-1R antagonist (e.g., anakinra) if the subject is refractory to tocilizumab.

In some embodiments, the human subject receives prophylactic treatment for tumor lysis syndrome (TLS) i.e. the subject is treated with prophylaxis for tumor lysis syndrome (TLS). Classification and grading of tumor lysis syndrome can be performed using methods known in the art, for example, as described in Howard et al. N Engl J Med 2011; 364:1844-54, and Coiffier et al., J Clin Oncol 2008; 26:2767-78. In some embodiments, prophylactic treatment of TLS comprises administering one or more uric acid reducing agents prior to administering the bispecific antibody i.e. the prophylaxis for TLS comprises administering one or more uric acid reducing agents prior to administration of the bispecific antibody. Exemplary uric acid reducing agents include allopurinol and rasburicase. Accordingly, in one embodiment, the prophylactic treatment of TLS comprises administering allopurinol and/or rasburicase. In some embodiments, the prophylactic treatment of TLS comprises administering allopurinol and/or rasburicase prior to administering the bispecific antibody. In one embodiment, allopurinol is administered 72 hours prior to the bispecific antibody. In some embodiments, rasburicase is initiated after administering allopurinol but prior to administering the bispecific antibody. Reassessment of the subject's TLS risk category can be performed prior to subsequent doses of the bispecific antibody. A subject is considered to be at low risk of TLS if all measurable lymph nodes have a largest diameter<5 cm and ALC<25×10⁹/L. A subject is considered to be at medium risk of TLS if any measurable lymph node has a largest diameter≥5 cm but<10 cm or ALC≥25×10⁹/L. A subject is considered to be at high risk of TLS if (a) any measurable lymph node has a largest diameter≥10 cm, or (b) ALC≥25×10⁹/L and any measurable lymph node has a largest diameter≥5 cm but<10 cm. Subjects with a lymphocyte count>100×10⁹/L are considered as high risk. In some embodiments, when the subject shows signs of TLS, supportive therapy, such as rasburicase and/or allopurinol, may be used.

In one embodiment, the bispecific antibody used in the methods described herein is administered subcutaneously, and thus is formulated in a pharmaceutical composition such that it is compatible with subcutaneous (s.c.) administration, i.e., having a formulation and/or concentration that allows pharmaceutical acceptable s.c. administration at the doses described herein. In some embodiments, subcutaneous administration is carried out by injection. For example, formulations for DuoBody-CD3xCD20 that are compatible with subcutaneous formulation and can be used in the methods described herein have been described previously (see, e.g., WO2019155008, which is incorporated herein by reference). In some embodiments, the bispecific antibody may be formulated using sodium acetate trihydrate, acetic acid, sodium hydroxide, sorbitol, polysorbate 80, and water for injection, and have a pH of 5.5 or about 5.5. In some embodiments, the bispecific antibody is provided as a 5 mg/mL or 60 mg/mL concentrate. In other embodiments, the desired dose of the bispecific antibody is reconstituted to a volume of about 1 mL for subcutaneous injection.

In one embodiment, a suitable pharmaceutical composition for the bispecific antibody can comprise the bispecific antibody, 20-40 mM acetate, 140-160 mM sorbitol, and a surfactant, such as polysorbate 80, and having a pH of 5.3-5.6. In some embodiments, the pharmaceutical formulation may comprise an antibody concentration in the range of 5-100 mg/mL, e.g., 48 or 60 mg/mL of the bispecific antibody, 30 mM acetate, 150 mM sorbitol, 0.04% w/v polysorbate 80, and have a pH of 5.5. Such a formulation may be diluted with, e.g., the formulation buffer to allow proper dosing and subcutaneous administration.

The volume of the pharmaceutical composition is appropriately selected to allow for subcutaneous administration of the antibody. For example, the volume to be administered is in the range of about 0.3 mL to about 3 mL, such as from 0.3 mL to 3 mL. The volume to be administered can be 0.5 mL, 0.8 mL, 1 mL, 1.2 mL, 1.5 ml, 1.7 mL, 2 mL, or 2.5 mL, or about 0.5 mL, about 0.8 mL, about 1 mL, about 1.2 mL, about 1.5 ml, about 1.7 mL, about 2 mL, or about 2.5 mL. Accordingly, in some embodiments, the volume to be administered is 0.5 mL or about 0.5 mL. In some embodiments, the volume to be administered is 0.8 mL or about 0.8 mL. In some embodiments, the volume to be administered is 1 mL or about 1 mL. In some embodiments, the volume to be administered is 1.2 mL or about 1.2 mL. In some embodiments, the volume to be administered is 1.5 mL or about 1.5 mL. In some embodiments, the volume to be administered is 1.7 mL or about 1.7 mL. In some embodiments, the volume to be administered is 2 mL or about 2 mL. In some embodiments, the volume to be administered is 2.5 mL or about 2.5 mL.

The methods (or uses of CD3xCD20 antibodies) described herein are for the treatment of human patients with CLL. It is understood that the methods described herein may be the first, or part of the first, treatment provided to such patients. However, patients may have been subjected to prior treatments for CLL and/or or Richter's syndrome. Prior treatments may include, but are not limited to, one or more of chemotherapy, immunotherapy, and targeted therapy, or combinations thereof. Most commonly, the standard of care for RS comprises treatments with a combination of cytotoxic chemotherapy and anti-CD20 monoclonal antibodies. It is understood that the methods described herein may also be used in combination with other treatments.

In one embodiment, the bispecific antibody used in the methods described herein comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences within the amino acid sequence of SEQ ID NO: 6, and the VL region comprises the CDR1, CDR2 and CDR3 sequences within the amino acid sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences within the amino acid sequence of SEQ ID NO: 13, and the VL region comprises the CDR1, CDR2 and CDR3 sequences within the amino acid sequence SEQ ID NO: 14.

CDR1, CDR2 and CDR3 regions can be identified from variable heavy and light chain regions using methods known in the art. The CDR regions from said variable heavy and light chain regions can be annotated according to IMGT (see Lefranc et al., Nucleic Acids Research 1999; 27:209-12 and Brochet. Nucl Acids Res 2008; 36:W503-8).

In some embodiments, the bispecific antibody comprises:
(i) a first binding arm comprising a first antigen-binding region which binds to human CD3ε (epsilon) and comprises VHCDR1, VHCDR2 and VHCDR3 the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) a second binding arm comprising a second antigen-binding region which binds to human CD20 and comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the bispecific antibody comprises:
(i) a first binding arm comprising a first antigen-binding region which binds to human CD3ε (epsilon) and comprises a VH region comprising the amino acid sequence of SEQ ID NO: 6, and a VL region comprising the amino acid sequence of SEQ ID NO: 7; and
(ii) a second binding arm comprising a second antigen-binding region which binds to human CD20 and comprises a VH region comprising the amino acid sequence of SEQ ID NO: 13, and a VL region comprising the amino acid sequence of SEQ ID NO: 14.

In some embodiments, the bispecific antibody is a full-length antibody. In some embodiments, the bispecific antibody comprises an inert Fc region. In one embodiment, the bispecific antibody is a full-length antibody and have an inert Fc region. In some embodiments, the first binding arm for CD3 is derived from a humanized antibody, e.g., from a full-length IgG1,λ (lambda) antibody such as H1L1 described in WO2015001085, which is incorporated herein by reference, and/or the second binding arm for CD20 is derived from a human antibody, e.g., from a full-length IgG1,κ (kappa) antibody such as clone 7D8 as described in WO2004035607, which kis incorporated herein by reference. The bispecific antibody may be produced from two half molecule antibodies, wherein each of the two half molecule antibodies comprises, e.g., the respective first and second binding arms set forth in SEQ ID NOs: 24 and 25, and SEQ ID NOs: 26 and 27. The half-antibodies may be produced in CHO cells and the bispecific antibodies generated by, e.g., Fab-arm exchange. In one embodiment, the bispecific antibody is a functional variant of DuoBody-CD3xCD20.

Accordingly, in some embodiments, the bispecific antibody comprises (i) a first binding arm comprising a first antigen-binding region which binds to human CD3ε (epsilon) and comprises a VH region comprising an amino acid sequence which is at least 85%, 90%, 95%, 96%, 97%, 98%, or 99% identical to SEQ ID NO: 6 or a VH region comprising the amino acid sequence of SEQ ID NO: 6, but with 1, 2, or 3 mutations (e.g., amino acid substitutions), and a VL region comprising an amino acid sequence which is at least 85%, 90%, 95%, 96%, 97%, 98%, or 99% identical to SEQ ID NO: 7 or a VL region comprising the amino acid sequence of SEQ ID NO: 7, but with 1, 2, or 3 mutations (e.g., amino acid substitutions); and
(ii) a second binding arm comprising a second antigen-binding region which binds to human CD20 and comprises a VH region comprising an amino acid sequence which is at least 85%, 90%, 95%, 98%, or 99% identical to SEQ ID NO: 13 or a VH region comprising the amino acid sequence of SEQ ID NO: 13, but with 1, 2, or 3 mutations (e.g., amino acid substitutions), and a VL region comprising an amino acid sequence which is at least 85%, 90%, 95%, 98%, or 99% identical to SEQ ID NO: 14 or a VL region comprising the amino acid sequence of SEQ ID NO: 14, but with 1, 2, or 3 mutations (e.g., amino acid substitutions).

In one embodiment, the bispecific antibody comprises:
(i) a first binding arm comprising a first antigen-binding region which binds to human CD3ε (epsilon) and comprises a heavy chain comprising the amino acid sequence of SEQ ID NO: 24, and a light chain comprising the amino acid sequence of SEQ ID NO: 25; and
(ii) a second binding arm comprising a second antigen-binding region which binds to human CD20 and comprises a VH region comprising the amino acid sequence of SEQ ID NO: 26, and a VL region comprising the amino acid sequence of SEQ ID NO: 27.

In some embodiments, the bispecific antibody comprises (i) a first binding arm comprising a first antigen-binding region which binds to human CD3ε (epsilon) and comprises a heavy chain comprising an amino acid sequence which is at least 85%, 90%, 95%, 98%, or 99% identical to SEQ ID NO: 24 or a heavy chain comprising the amino acid sequence of SEQ ID NO: 24, but with 1, 2, or 3 mutations (e.g., amino acid substitutions), and a light chain comprising an amino acid sequence which is at least 85%, 90%, 95%, 98%, or 99% identical to SEQ ID NO: 25 or a light chain region comprising the amino acid sequence of SEQ ID NO: 25, but with 1, 2, or 3 mutations (e.g., amino acid substitutions); and
(ii) a second binding arm comprising a second antigen-binding region which binds to human CD20 and comprises a heavy chain comprising an amino acid sequence which is at least 85%, 90%, 95%, 98%, or 99% identical to SEQ ID NO: 26 or a heavy chain comprising the amino acid sequence of SEQ ID NO: 26, but with 1, 2, or 3 mutations (e.g., amino acid substitutions), and a light chain comprising an amino acid sequence which is at least 85%, 90%, 95%, 98%, or 99% identical to SEQ ID NO: 27 or a light chain region comprising the amino acid sequence of SEQ ID NO: 27, but with 1, 2, or 3 mutations (e.g., amino acid substitutions).

Various constant regions or variants thereof may be used in the bispecific antibody. In one embodiment, the antibody comprises an IgG constant region, such as a human IgG1 constant region, e.g., a human IgG1 constant region as defined in SEQ ID NO: 15, or any other suitable IgG1 allotype. In some embodiments, the bispecific antibody is a full-length antibody with a human IgG1 constant region. In some embodiments, the first binding arm of the bispecific antibody is derived from a humanized antibody, preferably from a full-length IgG1,λ (lambda) antibody. In one embodiment, the first binding arm of the bispecific antibody is derived from a humanized antibody, e.g., from a full-length IgG1,λ (lambda) antibody, and thus comprises a λ light chain constant region. In some embodiments, the first binding arm comprises a λ light chain constant region as defined in SEQ ID NO: 22. In some embodiments, the second binding arm of the bispecific antibody is derived from a human antibody, preferably from a full-length IgG1,κ (kappa) antibody. In some embodiments the second binding arm of the bispecific antibody is derived from a human antibody, preferably from a full-length IgG1,κ (kappa) antibody, and thus may comprise a κ light chain constant region. In some embodiments, the second binding arm comprises a κ light chain constant region as defined in SEQ ID NO: 23. In a preferred embodiment, the first binding arm comprises a λ light chain constant region as defined in SEQ ID NO: 22 and the second binding arm comprises a κ light chain constant region as defined in SEQ ID NO: 23.

It is understood that the constant region portion of the bispecific antibody may comprise modifications that allow for efficient formation/production of bispecific antibodies and/or provide for an inert Fc region. Such modifications are well known in the art.

Different formats of bispecific antibodies are known in the art (reviewed by Kontermann, Drug Discov Today 2015; 20:838-47; MAbs, 2012; 4:182-97). Thus, the bispecific antibody used in the methods and uses described herein are not limited to any particular bispecific format or method of producing it. For example, bispecific antibodies may include, but are not limited to, bispecific antibodies with complementary CH3 domains to force heterodimerization, Knobs-into-Holes molecules (Genentech, WO9850431), CrossMAbs (Roche, WO2011117329), or electrostatically-matched molecules (Amgen, EP1870459 and WO2009089004; Chugai, US201000155133; Oncomed, WO2010129304).

Preferably, the bispecific antibody comprises an Fc-region comprising a first heavy chain with a first Fc sequence comprising a first CH3 region, and a second heavy chain with a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. Further details on these interactions and how they can be achieved are provided in e.g. WO2011131746 and WO2013060867 (Genmab), which are hereby incorporated by reference. In one embodiment, the bispecific antibody comprises in the first heavy chain (i) the amino acid L in the position corresponding to F405 in the human IgG1 heavy chain constant region of SEQ ID NO: 15, and comprises in the second heavy chain the amino acid R in the position corresponding to K409 in the human IgG1 heavy chain constant region of SEQ ID NO: 15, or vice versa.

Bispecific antibodies may comprise modifications in the Fc region to render the Fc region inert, or non-activating. Thus, in the bispecific antibodies disclosed herein, one or both heavy chains may be modified so that the antibody induces Fc-mediated effector function to a lesser extent relative to the bispecific antibody which does not have the modification. Fc-mediated effector function may be measured by determining Fc-mediated CD69 expression on T cells (i.e. CD69 expression as a result of CD3 antibody-mediated, Fcγ receptor-dependent CD3 crosslinking), by binding to Fcγ receptors, by binding to C1q, or by induction of Fc-mediated cross-linking of FcγRs. In particular, the heavy chain constant region sequence may be modified so that Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g. as described in Example 3 of WO2015001085. Modifications of the heavy and light chain constant region sequences may also result in reduced binding of C1q to said antibody. As compared to an unmodified antibody, the reduction may be by at least 70%, at least 80%, at least 90%, at least 95%, at least 97%, or 100%, and C1q binding may be determined, e.g., by ELISA. Further, the Fc region which may be modified so that the antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay. Examples of amino acid positions that may be modified, e.g., in an IgG1 isotype antibody, include positions L234 and L235. Thus, in one embodiment, the bispecific antibody may comprises a first heavy chain and a second heavy chain, and wherein in both the first heavy chain and the second heavy chain, the amino acid residues at the positions corresponding to positions L234 and L235 in a human IgG1 heavy chain according to Eu numbering are F and E, respectively. In addition, a D265A amino acid substitution can decrease binding to all Fcγ receptors and prevent ADCC (Shields et al., JBC 2001; 276:6591-604). Therefore, the bispecific antibody may comprise a first heavy chain and a second heavy chain, wherein in both the first heavy chain and the second heavy chain, the amino acid residue at the position corresponding to position D265 in a human IgG1 heavy chain according to Eu numbering is A.

In one embodiment, in the first heavy chain and second heavy chain of the bispecific antibody, the amino acids in the positions corresponding to positions L234, L235, and D265 in a human IgG1 heavy chain, are F, E, and A, respectively. An antibody having these amino acids at these positions is an example of an antibody having an inert Fc region, or a non-activating Fc region.

In some embodiments, the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively. In some embodiments, the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In a preferred embodiment, the bispecific antibody comprises a first heavy chain and a second heavy chain, wherein (i) in both the first and second heavy chains, the amino acids in the positions corresponding to positions L234, L235, and D265 in the human IgG1 heavy chain constant region of SEQ ID NO: 15 are F, E, and A, respectively, and (ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

With regard to the bispecific antibodies described herein, those which have the combination of three amino acid substitutions L234F, L235E and D265A and in addition the K409R or the F405L mutation, as described above, may be referred to with the suffix “FEAR” or “FEAL”, respectively.

An amino acid sequence of a wild type IgG1 heavy chain constant region may be identified herein as SEQ ID NO: 15. Consistent with the embodiments disclosed above, the bispecific antibody may comprise an IgG1 heavy chain constant region carrying the F405L substitution and may have the amino acid sequence set forth in SEQ ID NO: 17 and/or an IgG1 heavy chain constant region carrying the K409R substitution and may have the amino acid sequence set forth in SEQ ID NO: 18, and have further substitutions that render the Fc region inert or non-activating. Hence, in one embodiment, the bispecific antibody comprises a combination of IgG1 heavy chain constant regions, with the amino acid sequence of one of the IgG1 heavy chain constant regions carrying the L234F, L235E, D265A and F405L substitutions (e.g., as set forth in SEQ ID NO: 19) and the amino acid sequence of the other IgG1 heavy chain constant region carrying the L234F, L235E, D265A and K409R substitutions (e.g., as set forth in SEQ ID NO: 20). Thus, in some embodiments, the bispecific antibody comprises heavy chain constant regions comprising the amino acid sequences of SEQ ID NOs: 19 and 20.

In preferred embodiments, the bispecific antibody used in the methods and uses described herein comprises a first binding arm comprising a heavy chain and a light chain as defined in SEQ ID NOs: 24 and 25, respectively, and a second binding arm comprising a heavy chain and a light chain as defined in SEQ ID NOs: 26 and 27, respectively. Such an antibody can also be referred to herein as DuoBody-CD3xCD20. Also, variants of such antibodies are contemplated use in the methods and uses as described herein. In some embodiment, the bispecific antibody comprising a heavy chain and a light chain consisting of the amino acid sequences set forth in SEQ ID NOs: 24 and 25, respectively, and a heavy chain and a light chain consisting of the amino acid sequences set forth in SEQ ID NOs: 26 and 27, respectively. In some embodiments, the bispecific antibody is epcoritamab (CAS 2134641-34-0), or a biosimilar thereof.

### Kits

Also provided herein are kits which include a pharmaceutical composition containing a bispecific antibody which binds to CD3 and CD20 in accordance with the invention, such as DuoBody-CD3xCD20 or epcoritamab, and a pharmaceutically-acceptable carrier, in a therapeutically effective amount adapted for use in the methods described herein. The kits optionally also can include instructions, e.g., comprising administration schedules, to allow a practitioner (e.g., a physician, nurse, or patient) to administer the composition contained therein to administer the composition to a patient with CLL. The kit also can include a syringe.

Optionally, the kits include multiple packages of the single-dose (e.g., a dose between 12-60 mg, such as 12 mg, 24 mg, 36 mg, 48 mg, or 60 mg) pharmaceutical compositions each containing an effective amount of the bispecific antibody for a single administration in accordance with the methods described herein. Instruments or devices necessary for administering the pharmaceutical composition(s) also may be included in the kits. For instance, a kit may provide one or more pre-filled syringes containing an amount of the bispecific antibody.

### Further Embodiments

1. A method of treating Richter's syndrome (RS) in a human subject, the method comprising administering to the subject a bispecific antibody comprising:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose ranging from 12-60 mg in 28-day cycles.
   2. The method of embodiment 1, wherein the bispecific antibody is administered at a dose of 24 mg.
   3. The method of embodiment 1, wherein the bispecific antibody is administered at a dose of 48 mg.
   4. The method of any one of embodiments 1-3, wherein the bispecific antibody is administered once every week (weekly administration).
   5. The method of embodiment 4, wherein the weekly administration is performed for 2.5 28-day cycles.
   6. The method of embodiment 4 or 5, wherein after the weekly administration, the bispecific antibody is administered once every two weeks (biweekly administration).
   7. The method of embodiment 6, wherein the biweekly administration is performed for six 28-day cycles.
   8. The method of embodiment 6 or 7, wherein after the biweekly administration, the bispecific antibody is administered once every four weeks.
   9. The method of any one of embodiments 4-8, wherein prior to administering the first weekly dose of 12-60 mg, a priming dose of the bispecific antibody is administered in cycle 1 of the 28-day cycles.
   10. The method of embodiment 9, wherein the priming dose is administered two weeks prior to administering the first weekly dose of 12-60 mg.
   11. The method of embodiment 9 or 10, wherein the priming dose is in the range of 0.05-0.35 mg.
   12. The method of any one of embodiments 9-11, wherein said priming dose is 0.16 mg or about 0.16 mg.
   13. The method of any one of embodiments 9-12, wherein after administering the priming dose and prior to administering the first weekly dose of 12-60 mg, an intermediate dose of the bispecific antibody is administered.
   14. The method of embodiment 13, wherein the priming dose is administered on day 1 and the intermediate dose is administered on day 8 before the first weekly dose of 12-60 mg on days 15 and 22 of cycle 1.
   15. The method of embodiment 13 or 14, wherein said intermediate dose is in the range of 0.6-1.2 mg.
   16. The method of any one of embodiments 13-15, wherein said intermediate dose is 0.8 mg or about 0.8 mg.
   17. The method of any one of embodiments 13-16, wherein the bispecific antibody is administered in 28-day cycles, wherein:
a) in cycle 1, a priming dose is administered on day 1, an intermediate dose on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.
   18. The method of embodiment 17, wherein the full dose is 24 mg or about 24 mg.
   19. The method of embodiment 17, wherein the full dose is 48 mg or about 48 mg.
   20. The method of any one of embodiments 1-19, wherein the bispecific antibody is administered subcutaneously.
   21. The method of any one of embodiments 1-20, wherein the subject has clinical history of CLL/SLL with transformation toward aggressive lymphoma; e.g. of the DLBCL subtype.
   22. The method according to any one of embodiments 1-21, wherein the Richter's syndrome is of the DLBCL subtype.
   23. The method of any one of embodiments 1-22, wherein the subject has received one or more, such as at least two, prior lines of therapy for Chronic Lymphocytic Leukemia (CLL) and/or for Small Lymphocytic Lymphoma (SLL).
   24. The method of any one of embodiments wherein the prior lines of therapy for CLL and/or SLL comprise chemoimmunotherapy.
   25. The method of any one of embodiments wherein the prior lines of therapy for CLL and/or SLL comprise therapy with a targeted agent, such as BCL2 inhibitor or a BTK inhibitor.
   26. The method of any one of embodiments wherein the prior lines of therapy for CLL and/or SLL comprise CAR T-cell therapy.
   27. The method of any one of embodiments 1-23, wherein the subject has received prior therapy for Richter's syndrome, such as prior therapy selected from:
i) Rituximab in combination with cyclophosphamide, doxorubicin, vincristine, and prednisone (R-CHOP),
ii) Rituximab in combination with dexamethasone, cytarabine, and cisplatin (R-DHAP), and
iii) Venetoclax in combination with rituximab, etoposide, prednisone, vincristine, cyclophosphamide, and doxorubicin (VR-EPOCH).
   28. The method of any one of embodiments 1-27, wherein the subject achieves a complete metabolic response or a partial metabolic response.
   29. The method of any one of embodiments 1-27, wherein the subject achieves a complete response, a partial response, or stable disease.
   30. The method of any one of embodiments 1-26, wherein the subject receives epcoritamab as first-line therapy for Richter's syndrome.
   31. The method of any one of embodiments 1-30, wherein the subject achieves a complete metabolic response or a partial metabolic response.
   32. The method of any one of embodiments 1-30, wherein the subject achieves a complete response, a partial response, or stable disease.
   33. The method of any one of embodiments 1-26 and 30-32, the method being first-line therapy for Richter's syndrome.
   34. The method of any one of embodiments 21-29, wherein the subject has refractory and/or relapsed Richter's syndrome after receiving the said prior therapies.
   35. The method of any one of embodiments 1-34, wherein the subject is treated with prophylaxis for cytokine release syndrome (CRS).
   36. The method of embodiment 35, wherein the prophylaxis comprises administering a corticosteroid to the subject.
   37. The method of embodiment 35 or 36, wherein the corticosteroid is administered on the same day as the bispecific antibody.
   38. The method of embodiment 37, wherein the corticosteroid is further administered on the second, third, and fourth days after administering the bispecific antibody.
   39. The method of any one of embodiments 36-38, wherein the corticosteroid is prednisolone.
   40. The method of embodiment 39, wherein the prednisolone is administered at an intravenous dose of 100 mg, or equivalent thereof, including oral dose.
   41. The method of any one of embodiments 1-40, wherein the subject is administered premedication to reduce reactions to injections.
   42. The method of embodiment 41, wherein the premedication comprises an antihistamine.
   43. The method of embodiment 42, wherein the antihistamine is diphenhydramine.
   44. The method of embodiment 43, wherein the diphenhydramine is administered at an intravenous or oral dose of 50 mg, or equivalent thereof.
   45. The method of any one of embodiments 41-44, wherein the premedication comprises an antipyretic.
   46. The method of embodiment 45, wherein the antipyretic is acetaminophen.
   47. The method of embodiment 46, wherein the acetaminophen is administered at an oral dose of 560 to 1000 mg, or equivalent thereof.
   48. The method of any one of embodiments 41-47, wherein the premedication is administered on the same day as the bispecific antibody.
   49. The method of any one of embodiments 35-48, wherein the prophylaxis is administered during cycle 1.
   50. The method of any one of embodiments 41-49, wherein the premedication is administered during cycle 1.
   51. The method of embodiment 49 or 50, wherein the prophylaxis is administered during cycle 2 when the subject experiences CRS greater than grade 1 after the last administration of the bispecific antibody in cycle 1.
   52. The method of embodiment 51, wherein the prophylaxis is continued in a subsequent cycle, when in the last administration of the bispecific antibody of the previous cycle, the subject experiences CRS greater than grade 1.
   53. The method of any one of embodiments 41-52, wherein the premedication is administered during cycle 2.
   54. The method of embodiment 53, wherein the premedication is administered during subsequent cycles.
   55. The method of any one of embodiments 1-54, wherein the subject is administered antibiotics if the subject develops Grade 1 CRS.
   56. The method of any one of embodiments 1-54, wherein the subject is administered a vasopressor if the subject develops Grade 2 or Grade 3 CRS.
   57. The method of any one of embodiments 1-54, wherein the subject is administered at least two vasopressors if the subject develops Grade 4 CRS.
   58. The method of any one of embodiments 1-57, wherein the subject is administered tocilizumab if the subject develops Grade 2, Grade 3, or Grade 4 CRS.
   59. The method of embodiment 58, wherein the subject is further administered a steroid.
   60. The method of embodiment 59, wherein the steroid is dexamethasone.
   61. The method of embodiment 59, wherein the steroid is methylprednisolone.
   62. The method of any one of embodiments 58-61, wherein tocilizumab is switched to an anti-IL-6 antibody (e.g., siltuximab) if the subject is refractory to tocilizumab.
   63. The method of any one of embodiments 58-61, wherein tocilizumab is switched to an IL-1R antagonist (e.g., anakinra) if the subject is refractory to tocilizumab.
   64. The method of any one of embodiments 1-63, wherein the subject is treated with prophylaxis for tumor lysis syndrome (TLS).
   65. The method of embodiment 64, wherein the prophylaxis for TLS comprises administering one or more uric acid reducing agents prior to administration of the bispecific antibody.
   66. The method of embodiment 65, wherein the one or more uric acid reducing agents comprise rasburicase and/or allopurinol.
   67. The method of any one of embodiments 1-66, wherein the subject achieves a complete response, a partial response, or stable disease.
   68. The method of any one of embodiments 1-67, wherein:
(i) the first antigen-binding region comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.
   69. The method of any one of embodiments 1-68, wherein:
(i) the first antigen-binding region comprises a VH region comprising the amino acid sequence of SEQ ID NO: 6, and the VL region comprising the amino acid sequence of SEQ ID NO: 7; and
(ii) the second antigen-binding region comprises a VH region comprising the amino acid sequence of SEQ ID NO: 13, and the VL region comprising the amino acid sequence of SEQ ID NO: 14.
   70. The method of any one of embodiments 1-68, wherein the first binding arm of the bispecific antibody is derived from a humanized antibody, preferably from a full-length IgG1,λ (lambda) antibody.
   71. The method of embodiment 70, wherein the first binding arm of the bispecific antibody comprises a λ light chain constant region comprising the amino acid sequence set forth in SEQ ID NO: 22.
   72. The method of any one of embodiments 1-71, wherein the second binding arm of the bispecific antibody is derived from a human antibody, preferably from a full-length IgG1,κ (kappa) antibody.
   73. The method of embodiment 72, wherein the second binding arm comprises a κ light chain constant region comprising the amino acid sequence set forth in SEQ ID NO: 23.
   74. The method of any one of embodiments 1-73, wherein the bispecific antibody is a full-length antibody with a human IgG1 constant region.
   75. The method of any one of embodiments 1-74, wherein the bispecific antibody comprises an inert Fc region.
   76. The method of any one of embodiments 1-75, wherein the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively.
   77. The method of any one of embodiments 1-76, wherein the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
   78. The method of any one of embodiments 1-77, wherein the bispecific antibody comprises a first heavy chain and a second heavy chain, wherein
(i) in both the first and second heavy chains, the amino acids in the positions corresponding to positions L234, L235, and D265 in the human IgG1 heavy chain constant region of SEQ ID NO: 15 are F, E, and A, respectively, and
(ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
   79. The method of embodiment 78, wherein the bispecific antibody comprises heavy chain constant regions comprising the amino acid sequences of SEQ ID NOs: 19 and 20.
   80. The method of any one of embodiments 1-79, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.
   81. The method of any one of embodiments 1-80, wherein the bispecific antibody comprises a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.
   82. The method of any one of embodiments 1-81, wherein the bispecific antibody is epcoritamab, or a biosimilar thereof.
   1a. A bispecific antibody comprising:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
for use in treating Richter's syndrome (RS) in a human subject, the treatment comprising administering to the subject the bispecific antibody at a dose ranging from 12-60 mg in 28-day cycles.
   2a. The bispecific antibody for use of embodiment 1a, wherein the bispecific antibody is administered at a dose of 24 mg.
   3a. The bispecific antibody for use of embodiment 1a, wherein the bispecific antibody is administered at a dose of 48 mg.
   4a. The bispecific antibody for use of any one of embodiments 1-3a, wherein the bispecific antibody is administered once every week (weekly administration).
   5a. The bispecific antibody for use of embodiment 4a, wherein the weekly administration is performed for 2.5 28-day cycles.
   6a. The bispecific antibody for use of embodiment 4a or 5a, wherein after the weekly administration, the bispecific antibody is administered once every two weeks (biweekly administration).
   7a. The bispecific antibody for use of embodiment 6a, wherein the biweekly administration is performed for six 28-day cycles.
   8a. The bispecific antibody for use of embodiment 6a or 7a, wherein after the biweekly administration, the bispecific antibody is administered once every four weeks.
   9a. The bispecific antibody for use of any one of embodiments 4-8a, wherein prior to administering the first weekly dose of 12-60 mg, a priming dose of the bispecific antibody is administered in cycle 1 of the 28-day cycles.
   10a. The bispecific antibody for use of embodiment 9a, wherein the priming dose is administered two weeks prior to administering the first weekly dose of 12-60 mg.
   11a. The bispecific antibody for use of embodiment 9a or 10a, wherein the priming dose is in the range of 0.05-0.35 mg.
   12a. The bispecific antibody for use of any one of embodiments 9-11a, wherein said priming dose is 0.16 mg or about 0.16 mg.
   13a. The bispecific antibody for use of any one of embodiments 9-12a, wherein after administering the priming dose and prior to administering the first weekly dose of 12-60 mg, an intermediate dose of the bispecific antibody is administered.
   14a. The bispecific antibody for use of embodiment 13a, wherein the priming dose is administered on day 1 and the intermediate dose is administered on day 8 before the first weekly dose of 12-60 mg on days 15 and 22 of cycle 1.
   15a. The bispecific antibody for use of embodiment 13a or 14a, wherein said intermediate dose is in the range of 0.6-1.2 mg.
   16a. The bispecific antibody for use of any one of embodiments 13-15a, wherein said intermediate dose is 0.8 mg or about 0.8 mg.
   17a. The bispecific antibody for use of any one of embodiments 13-16a, wherein the bispecific antibody is administered in 28-day cycles, wherein:
a) in cycle 1, a priming dose is administered on day 1, an intermediate dose on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.
   18a. The bispecific antibody for use of embodiment 17a, wherein the full dose is 24 mg or about 24 mg.
   19a. The bispecific antibody for use of embodiment 17a, wherein the full dose is 48 mg or about 48 mg.
   20a. The bispecific antibody for use of any one of embodiments 1-19a, wherein the bispecific antibody is administered subcutaneously.
   21a. The bispecific antibody for use of any one of embodiments 1-20a, wherein the subject has clinical history of CLL/SLL with transformation toward aggressive lymphoma; e.g. of the DLBCL subtype.
   22a. The bispecific antibody for use according to any one of embodiments 1-21a, wherein the Richter's syndrome is of the DLBCL subtype.
   23a. The bispecific antibody for use of any one of embodiments 1-22a, wherein the subject has received one or more, such as at least two, prior lines of therapy for Chronic Lymphocytic Leukemia (CLL) and/or for Small Lymphocytic Lymphoma (SLL).
   24a. The bispecific antibody for use of any one of embodiments wherein the prior lines of therapy for CLL and/or SLL comprise chemoimmunotherapy.
   25a. The bispecific antibody for use of any one of embodiments wherein the prior lines of therapy for CLL and/or SLL comprise therapy with a targeted agent, such as BCL2 inhibitor or a BTK inhibitor.
   26a. The bispecific antibody for use of any one of embodiments wherein the prior lines of therapy for CLL and/or SLL comprise CAR T-cell therapy.
   27a. The bispecific antibody for use of any one of embodiments 1-23a, wherein the subject has received prior therapy for Richter's syndrome, such as prior therapy selected from:
i) Rituximab in combination with cyclophosphamide, doxorubicin, vincristine, and prednisone (R-CHOP),
ii) Rituximab in combination with dexamethasone, cytarabine, and cisplatin (R-DHAP), and
iii) Venetoclax in combination with rituximab, etoposide, prednisone, vincristine, cyclophosphamide, and doxorubicin (VR-EPOCH).
   28a. The bispecific antibody for use of any one of embodiments 1-27a, wherein the subject achieves a complete metabolic response or a partial metabolic response.
   29a. The bispecific antibody for use of any one of embodiments 1-27a, wherein the subject achieves a complete response, a partial response, or stable disease.
   30a. The bispecific antibody for use of any one of embodiments 1-26a, wherein the subject receives epcoritamab as first-line therapy for Richter's syndrome.
   31a. The bispecific antibody for use of any one of embodiments 1-30a, wherein the subject achieves a complete metabolic response or a partial metabolic response.
   32a. The bispecific antibody for use of any one of embodiments 1-30a, wherein the subject achieves a complete response, a partial response, or stable disease.
   33a. The bispecific antibody for use of any one of embodiments 1-26a and 30-32a, the method being first-line therapy for Richter's syndrome.
   34a. The bispecific antibody for use of any one of embodiments 21-29a, wherein the subject has refractory and/or relapsed Richter's syndrome after receiving the said prior therapies.
   35a. The bispecific antibody for use of any one of embodiments 1-34a, wherein the subject is treated with prophylaxis for cytokine release syndrome (CRS).
   36a. The bispecific antibody for use of embodiment 35a, wherein the prophylaxis comprises administering a corticosteroid to the subject.
   37a. The bispecific antibody for use of embodiment 35a or 36a, wherein the corticosteroid is administered on the same day as the bispecific antibody.
   38a. The bispecific antibody for use of embodiment 37a, wherein the corticosteroid is further administered on the second, third, and fourth days after administering the bispecific antibody.
   39a. The bispecific antibody for use of any one of embodiments 36-38a, wherein the corticosteroid is prednisolone.
   40a. The bispecific antibody for use of embodiment 39a, wherein the prednisolone is administered at an intravenous dose of 100 mg, or equivalent thereof, including oral dose.
   41a. The bispecific antibody for use of any one of embodiments 1-40a, wherein the subject is administered premedication to reduce reactions to injections.
   42a. The bispecific antibody for use of embodiment 41a, wherein the premedication comprises an antihistamine.
   43a. The bispecific antibody for use of embodiment 42a, wherein the antihistamine is diphenhydramine.
   44a. The bispecific antibody for use of embodiment 43a, wherein the diphenhydramine is administered at an intravenous or oral dose of 50 mg, or equivalent thereof.
   45a. The bispecific antibody for use of any one of embodiments 41-44a, wherein the premedication comprises an antipyretic.
   46a. The bispecific antibody for use of embodiment 45a, wherein the antipyretic is acetaminophen.
   47a. The bispecific antibody for use of embodiment 46a, wherein the acetaminophen is administered at an oral dose of 560 to 1000 mg, or equivalent thereof.
   48a. The bispecific antibody for use of any one of embodiments 41-47a, wherein the premedication is administered on the same day as the bispecific antibody.
   49a. The bispecific antibody for use of any one of embodiments 35-48a, wherein the prophylaxis is administered during cycle 1.
   50a. The bispecific antibody for use of any one of embodiments 41-49a, wherein the premedication is administered during cycle 1.
   51a. The bispecific antibody for use of embodiment 49a or 50a, wherein the prophylaxis is administered during cycle 2 when the subject experiences CRS greater than grade 1 after the last administration of the bispecific antibody in cycle 1.
   52a. The bispecific antibody for use of embodiment 51a, wherein the prophylaxis is continued in a subsequent cycle, when in the last administration of the bispecific antibody of the previous cycle, the subject experiences CRS greater than grade 1.
   53a. The bispecific antibody for use of any one of embodiments 41-52a, wherein the premedication is administered during cycle 2.
   54a. The bispecific antibody for use of embodiment 53a, wherein the premedication is administered during subsequent cycles.
   55a. The bispecific antibody for use of any one of embodiments 1-54a, wherein the subject is administered antibiotics if the subject develops Grade 1 CRS.
   56a. The bispecific antibody for use of any one of embodiments 1-54a, wherein the subject is administered a vasopressor if the subject develops Grade 2 or Grade 3 CRS.
   57a. The bispecific antibody for use of any one of embodiments 1-54a, wherein the subject is administered at least two vasopressors if the subject develops Grade 4 CRS.
   58a. The bispecific antibody for use of any one of embodiments 1-57a, wherein the subject is administered tocilizumab if the subject develops Grade 2, Grade 3, or Grade 4 CRS.
   59a. The bispecific antibody for use of embodiment 58a, wherein the subject is further administered a steroid.
   60a. The bispecific antibody for use of embodiment 59a, wherein the steroid is dexamethasone.
   61a. The bispecific antibody for use of embodiment 59a, wherein the steroid is methylprednisolone.
   62a. The bispecific antibody for use of any one of embodiments 58-61a, wherein tocilizumab is switched to an anti-IL-6 antibody (e.g., siltuximab) if the subject is refractory to tocilizumab.
   63a. The bispecific antibody for use of any one of embodiments 58-61a, wherein tocilizumab is switched to an IL-1R antagonist (e.g., anakinra) if the subject is refractory to tocilizumab.
   64a. The bispecific antibody for use of any one of embodiments 1-63a, wherein the subject is treated with prophylaxis for tumor lysis syndrome (TLS).
   65a. The bispecific antibody for use of embodiment 64a, wherein the prophylaxis for TLS comprises administering one or more uric acid reducing agents prior to administration of the bispecific antibody.
   66a. The bispecific antibody for use of embodiment 65a, wherein the one or more uric acid reducing agents comprise rasburicase and/or allopurinol.
   67a. The bispecific antibody for use of any one of embodiments 1-66a, wherein the subject achieves a complete response, a partial response, or stable disease.
   68a. The bispecific antibody for use of any one of embodiments 1-67a, wherein:
(i) the first antigen-binding region comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.
   69a. The bispecific antibody for use of any one of embodiments 1-68a, wherein:
(i) the first antigen-binding region comprises a VH region comprising the amino acid sequence of SEQ ID NO: 6, and the VL region comprising the amino acid sequence of SEQ ID NO: 7; and
(ii) the second antigen-binding region comprises a VH region comprising the amino acid sequence of SEQ ID NO: 13, and the VL region comprising the amino acid sequence of SEQ ID NO: 14.
   70a. The bispecific antibody for use of any one of embodiments 1-68a, wherein the first binding arm of the bispecific antibody is derived from a humanized antibody, preferably from a full-length IgG1,λ (lambda) antibody.
   71a. The bispecific antibody for use of embodiment 70a, wherein the first binding arm of the bispecific antibody comprises a λ light chain constant region comprising the amino acid sequence set forth in SEQ ID NO: 22.
   72a. The bispecific antibody for use of any one of embodiments 1-71a, wherein the second binding arm of the bispecific antibody is derived from a human antibody, preferably from a full-length IgG1,κ (kappa) antibody.
   73a. The bispecific antibody for use of embodiment 72a, wherein the second binding arm comprises a κ light chain constant region comprising the amino acid sequence set forth in SEQ ID NO: 23.
   74a. The bispecific antibody for use of any one of embodiments 1-73a, wherein the bispecific antibody is a full-length antibody with a human IgG1 constant region.
   75a. The bispecific antibody for use of any one of embodiments 1-74a, wherein the bispecific antibody comprises an inert Fc region.
   76a. The bispecific antibody for use of any one of embodiments 1-75a, wherein the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively.
   77a. The bispecific antibody for use of any one of embodiments 1-76a, wherein the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
   78a. The bispecific antibody for use of any one of embodiments 1-77a, wherein the bispecific antibody comprises a first heavy chain and a second heavy chain, wherein
(i) in both the first and second heavy chains, the amino acids in the positions corresponding to positions L234, L235, and D265 in the human IgG1 heavy chain constant region of SEQ ID NO: 15 are F, E, and A, respectively, and
(ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
   79a. The bispecific antibody for use of embodiment 78a, wherein the bispecific antibody comprises heavy chain constant regions comprising the amino acid sequences of SEQ ID NOs: 19 and 20.
   80a. The bispecific antibody for use of any one of embodiments 1-79a, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.
   81a. The bispecific antibody for use of any one of embodiments 1-80a, wherein the bispecific antibody comprises a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.
   82a. The bispecific antibody for use of any one of embodiments 1-81a, wherein the bispecific antibody is epcoritamab, or a biosimilar thereof.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### DuoBody-CD3xCD20

DuoBody-CD3xCD20 is a bsAb recognizing the T-cell antigen CD3 and the B-cell antigen CD20. DuoBody-CD3xCD20 triggers potent T-cell-mediated killing of CD20-expressing cells. DuoBody-CD3xCD20 has a regular IgG1 structure.

Two parental antibodies, IgG1-CD3-FEAL, a humanized IgG1k, CD3ε-specific antibody having heavy and light chain sequences as listed in SEQ ID NOs: 24 and 25, respectively, and IgG1-CD20-FEAR, derived from human IgG1κ CD20-specific antibody 7D8 having heavy and light chain sequences as listed in SEQ ID NOs: 26 and 27, respectively, were manufactured as separate biological intermediates. Each parental antibody contains one of the complementary mutations in the CH3 domain required for the generation of DuoBody molecules (F405L and K409R, respectively). The parental antibodies comprised three additional mutations in the Fc region (L234F, L235E and D265A; FEA). The parental antibodies were produced in mammalian Chinese hamster ovary (CHO) cell lines using standard suspension cell cultivation and purification technologies. DuoBody-CD3xCD20 was subsequently manufactured by a controlled Fab-arm exchange (cFAE) process (Labrijn et al. 2013, Labrijn et al. 2014, Gramer et al. 2013). The parental antibodies are mixed and subjected to controlled reducing conditions. This leads to separation of the parental antibodies that, under re-oxidation, re-assemble. This way, highly pure preparations of DuoBody-CD3xCD20 (˜ 93-95%) were obtained. After further polishing/purification, final product was obtained, close to 100% pure. The DuoBody-CD3xCD20 concentration was measured by absorbance at 280 nm, using the theoretical extinction coefficient ε=1.597 mL·mg⁻¹cm⁻¹. The product has received the international proprietary name of epcoritamab.

Epcoritamab is prepared (5 mg/mL or 60 mg/mL) as a sterile clear colorless to slightly yellow solution supplied as concentrate for solution for subcutaneous (SC) injection. Epcoritamab contains buffering and tonicifying agents. All excipients and amounts thereof in the formulated product are pharmaceutically acceptable for subcutaneous injection products. Appropriate doses are reconstituted to a volume of about 1 mL for subcutaneous injection.

### Example 1: A Phase 1b/2, Open-Label, Safety and Efficacy Study of Epcoritamab in Relapsed/Refractory Chronic Lymphocytic Leukemia and Richter's Syndrome (RS)

The purpose of this Phase 1b/2 study is to evaluate the safety and preliminary efficacy of single agent epcoritamab in subjects with Richter's syndrome (RS). The study is an open-label, 2-part (dose escalation and expansion), multicenter study conducted to evaluate the safety, tolerability, PK, pharmacodynamics, immunogenicity, and preliminary efficacy of single agent epcoritamab in subjects aged 18 years or older with relapsed and/or refractory (R/R) chronic lymphocytic leukemia (CLL) or Richter's syndrome.

The trial includes 2 parts: dose escalation (Part 1) and expansion (Part 2). The overall study design is further disclosed in WO 2021/224499. The disclosure provides objectives of the dose escalation part, including identification of the recommended phase 2 dose (RP2D) and maximum tolerated dose (MTD). Epcoritamab was studied at 2 full dose levels: 24 mg and 48 mg. A step-up dosing regimen is applied: 0.16 mg/0.8 mg/24 mg and 0.16 mg/0.8 mg/48 mg (priming/intermediate/full dose)

An expansion cohort (Expansion cohort 1) investigating treatment of relapsed or refractory chronic lymphocytic leukemia (R/R CLL) is also disclosed in WO 2021/224499.

Finally, the disclosure in WO 2021/224499 includes preliminary results from the dose escalation phase data suggesting that epcoritamab is well tolerated in patients with R/R CLL at dose levels up to 48 mg and has encouraging clinical activity in patients with high-risk disease.

### Objectives

### Expansion Cohort 2, Richter's Syndrome

The primary objective of the present arm of expansion cohort 2 is to assess the preliminary efficacy of epcoritamab in subjects with Richter's syndrome (endpoint: ORR).

Secondary objectives of expansion cohort 2 include evaluating the preliminary efficacy of epcoritamab (endpoints: Overall response rate (ORR), duration of response (DOR), complete remission (CR), time to response (TTR), progression-free survival (PFS), overall survival (OS), and time to next anti-cancer therapy (TTNT)), assessing the MRD status in peripheral blood and bone marrow (endpoint: incidence of undetectable MRD), evaluating the safety and tolerability of epcoritamab (endpoints: incidence and severity of adverse events (AEs), serious adverse events (SAEs), cytokine release syndrome (CRS), immune effector cell-associated neurotoxicity syndrome (ICANs), and tumor lysis syndrome (TLS), and incidence of dose interruption, dose delay, and dose intensity), establishing the pharmacokinetics (PK) and pharmacodynamic profiles of epcoritamab (endpoints: PK parameters and pharmacodynamic parameters), and evaluating immunogenicity of epcoritamab (endpoint: incidence of anti-drug antibodies (ADAs) to epcoritamab).

Exploratory objectives of the expansion part include evaluating biomarkers predictive of clinical response to epcoritamab (endpoints: expression of CD20 and evaluation of immune populations, phenotype, and function, and blood).

### Study Design Overview

### Expansion Cohort 2, Richter's Syndrome

The expansion part enrolls approximately 70 subjects with Richter's syndrome (RS).

Epcoritamab is administered as a subcutaneous (SC) injection in 4-week cycles (i.e., 28 days), as shown below, until one or more of the discontinuation criteria are met:
Cycle 1-3: Days 1, 8, 15 and 22 (QW)
Cycle 4-9: Days 1 and 15 (Q2W)
Cycle 10 and beyond: Day 1 (Q4W)
   A step-up dosing method is used to mitigate the potential for CRS: priming dose of 0.16 mg on Cycle 1 Day 1, followed by intermediate dose of 0.8 mg on Cycle 1 Day 8, then full dose of 48 mg on Cycle 1 Day 15 and Day 22, and full dose of 48 mg in subsequent cycles.

The primary efficacy endpoint of the expansion part is ORR as assessed using the iwCLL 2018 criteria (Table 2). Secondary efficacy endpoints include DOR, CR, TTR, PFS, OS, and TTNT. Incidence of MRD negative status/undetectable MRD is also evaluated as a secondary efficacy endpoint. MRD assessment indicates how many cancer cells still remain in a subject who is in remission either during or after treatment has been implemented. Safety endpoints in the expansion part include the incidence and severity of AEs/SAEs, incidence and severity of tumor lysis syndrome (TLS), immune effector cell-associated neurotoxicity syndrome (ICANS) and CRS, and incidence of treatment interruption and delay.

### Inclusion Criteria

1. Subject must sign an ICF, prior to any screening procedures, indicating that he or she understands the purpose of and procedures required for the trial and are willing to participate in the trial prior to any other trial related assessments or procedures. Where required by local or country specific regulations, each subject must sign a separate ICF if he or she agrees to provide samples for genomic biomarker analysis (DNA). If a subject refuses to consent to DNA research in these specific regions, the subject is still eligible to participate in the trial.
2. Subjects must be at least 18 years of age.
3. Must have a clinical history of CLL/SLL with biopsy-proven transformation toward aggressive lymphoma (ie, DLBCL subtype).
4. Deemed as ineligible for chemoimmunotherapy at investigator's discretion or refuse to receive intensive chemotherapy.
5. Must have measurable disease as determined by both
a. A fluorodeoxyglucose (FDG)-positron emission tomography (PET) CT scan demonstrating positive lesion compatible with CT (or MRI)-defined anatomical tumor sites; and
b. A CT scan (or MRI) with involvement of ≥2 clearly demarcated lesions/nodes with long axis>1.5 cm and short axis>1.0 cm or 1 clearly demarcated lesion/node with a long axis>2.0 cm and a short axis≥1.0 cm.
   6. ECOG performance status score of 0, 1 or 2
   7. Evidence of CD20 positivity at screening
   8. Has acceptable laboratory parameters as follows:

TABLE 4

| Parameter | | Result |
|---|---|---|
| a. | Creatine clearance or serum | >45 mL/min (Cockcroft-Gault) or serum |
| | creatine | creatinine ≤1.5 times the upper limit of normal |
| | | (×ULN) |
| b. | Serum alanine transaminase | ≤3 × ULN |
| | (ALT) | |
| c. | Serum aspartate transaminase | ≤3 × ULN |
| | (AST) | |
| d. | Bilirubin | ≤1.5 × ULN unless due to Gilbert syndrome |
| | | Note: Subjects with Gilbert's syndrome may be |
| | | included if total bilirubin is ≤3 × ULN and direct |
| | | bilirubin is ≤1.5 × ULN |
| e. | Hemoglobin | ≥9.0 g/dL unless anemia is due to marrow |
| | | involvement of CLL |
| | | Note: Blood transfusion may be administered |
| | | during screening to meet this requirement |
| f. | Absolute neutrophil count | ≥1.0 × 10⁹/L (1000/μL) unless neutropenia is due |
| | | to bone marrow involvement of CLL. |
| | | Note: Growth factor support is allowed in case of |
| | | bone marrow involvement. |
| g. | Platelet count | ≥30 × 10⁹/L (30,000/μL) |
| | | Note: Transfusion may be administered during |
| | | screening to meet this requirement. |
| h. | Coagulation status | PT/INR/aPTT ≤1.5 × ULN |

8. Received a cumulative dose of corticosteroids less than the equivalent of 250 mg of prednisone within the 2-week period before the first dose of epcoritamab
9. Subject must have availability of fresh bone marrow material at screening.
10. A woman with reproductive potential must agree to use adequate contraception during the trial, and for 12 months after the last administration of epcoritamab. Adequate contraception is defined as highly effective methods of contraception.
11. A woman of childbearing potential must have a negative serum (beta-hCG) pregnancy test at screening and a negative serum or urine pregnancy test before treatment administration on Day 1 of every cycle.
12. A woman must agree not to donate eggs (ova, oocytes) for the purposes of assisted reproduction during the entire trial, until 12 months after last treatment.
13. A man who is sexually active with a woman of childbearing potential and has not had a vasectomy must agree to use a barrier method of birth control, eg either condom with spermicidal foam/gel/film/cream/suppository and partner with occlusive cap (diaphragm or cervical/vault caps) with spermicidal foam/gel/film/cream/suppository, and all men must also not donate sperm during the trial and 12 months after receiving the last dose of epcoritamab.
14. Subject must be willing and able to adhere to the prohibitions and restrictions specified in this protocol.

### Exclusion Criteria

1. Diagnosis of Richter's syndrome not of the DLBCL subtype such as Hodgkin's lymphoma, prolymphocytic leukemia.
2. Subject received autologous HSCT within 3 months prior to the first dose of epcoritamab.
3. Subject received more than 1 prior line of therapy for RS.
4. Subject received prior treatment with a CD3×CD20 bispecific antibody
5. Subject received any prior allogeneic HSCT or solid organ transplantation
6. Subject received treatment with an anti-cancer agent, e.g.
a. Small molecules such as BTK inhibitor, BCL2 inhibitor, or PI3K inhibitor within 5 half-lives prior to the first dose of epcoritamab; or
b. Anti-CD20 mAb or chemotherapy within 2 weeks prior to the first dose of epcoritamab; or
c. Radio-conjugated or toxin conjugated antibody or CAR-T cell therapy within 4 weeks or 5 half-lives, whichever is shorter, prior to the first dose of epcoritamab
d. Subject received treatment with an investigational drug, within 4 weeks or 5 half-lives, whichever is shorter prior to the first dose of epcoritamab.
   7. Subject has autoimmune disease or other diseases that require permanent or high-dose immunosuppressive therapy
   8. Subject has clinically significant cardiac disease including but not limited to:
a. Unstable or uncontrolled disease/condition related to or affecting cardiac function, eg, unstable angina, congestive heart failure grade III or IV as classified by the New York Heart Association (see Appendix 3), cardiac arrhythmia (CTCAE v5.0 grade 2 or higher) or clinically significant electrocardiogram (ECG) abnormalities
b. Myocardial infarction, intracranial bleed, or stroke within the past 6 months
c. Screening 12-lead ECG showing a baseline QT interval as corrected by Fridericia's formula (QTcF)>480
   9. Subject received vaccination with live vaccines within 28 days prior to the first dose of epcoritamab
   10. Subject has toxicities from previous anti-cancer therapies that have not resolved to baseline levels or to Grade 1 or less except for alopecia and peripheral neuropathy
   11. Subject has known CNS involvement at screening
   12. Subject has known past or current malignancy other than inclusion diagnosis, except for:
a. Cervical carcinoma of Stage 1B or less
b. Non-invasive basal cell or squamous cell skin carcinoma
c. Non-invasive, superficial bladder cancer
d. Prostate cancer with a current PSA level<0.1 ng/mL
e. Any curable cancer with a CR of >2 years duration
   13. Subject has suspected allergies, hypersensitivity, or intolerance to epcoritamab or its excipients
   14. Subject has had major surgery within 4 weeks prior to enrollment.
   15. Subject has known history/positive serology for hepatitis B (unless immune due to vaccination or resolved natural infection or unless passive immunization due to immunoglobulin therapy):
a. Positive test for antibodies to the hepatitis B core antigen (anti-HBc) AND
b. Negative test for antibodies to the hepatitis B surface antigen (anti-HBs).
   16. Known medical history or ongoing hepatitis C infection that has not been cured.
   17. Known history of seropositivity for HIV infection. Note: HIV testing is required at screening only if required per local health authorities or institutional standards.
   18. Subject is a woman who is pregnant or breast-feeding, or who is planning to become pregnant while enrolled in this trial or within 12 months after the last dose of epcoritamab.
   19. Subject is a man who plans to father a child while enrolled in this trial or within 12 months after the last dose of epcoritamab.
   20. Subject has any condition for which participation would note in the best interest of the subject (e.g., compromise the well-being) or that could prevent, limit, or confound the protocol-specified assessments.
   21. Subject has uncontrolled intercurrent illness, such as ongoing or active infection requiring intravenous antibiotics treatment at the time of enrollment or within the previous 2 weeks prior to the first dose of epcoritamab.

### Premedication and CRS Prophylaxis

Premedication with corticosteroids, antihistamines, and antipyretics is mandatory as described in Table 5. For each dose of epcoritamab in Cycle 1, 4 consecutive days of corticosteroids are mandatory to prevent/reduce severity of symptoms from potential CRS as described in Table 5. For administration of epcoritamab in Cycle 2 and beyond, CRS prophylaxis with corticosteroids is optional. Corticosteroid administration can be either intravenous or oral route with recommended dose or equivalent.

TABLE 5
Prophylactic corticosteroid administration pre- and post-epcoritamab administration

| | | | Corticosteroids** | Antihistamines | Antipyretics |
|---|---|---|---|---|---|
| Cycle | 1^{st} | Day | Prednisolone 100 mg IV | Diphenhydramine | Paracetamol |
| 1 | epcoritamab | 01* | (or equivalent including | 50 mg IV or oral | (acetaminophen) |
| | administration | | oral dose) | (PO) (or equivalent) | 650 to 1000 mg PO |
| | (priming | | | | (or equivalent) |
| | dose) | Day | Prednisolone 100 mg IV | | |
| | | 02 | (or equivalent including | | |
| | | | oral dose) | | |
| | | Day | Prednisolone 100 mg IV | | |
| | | 03 | (or equivalent including | | |
| | | | oral dose) | | |
| | | Day | Prednisolone 100 mg IV | | |
| | | 04 | (or equivalent including | | |
| | | | oral dose) | | |
| | 2^{nd} | Day | Prednisolone 100 mg IV | Diphenhydramine | Paracetamol |
| | epcoritamab | 08* | (or equivalent including | 50 mg IV or oral | (acetaminophen) |
| | administration | | oral dose) | (PO) (or equivalent) | 650 to 1000 mg PO |
| | (intermediate | | | | (or equivalent) |
| | dose) | Day | Prednisolone 100 mg IV | | |
| | | 09 | (or equivalent including | | |
| | | | oral dose) | | |
| | | Day | Prednisolone 100 mg IV | | |
| | | 10 | (or equivalent including | | |
| | | | oral dose) | | |
| | | Day | Prednisolone 100 mg IV | | |
| | | 11 | (or equivalent including | | |
| | | | oral dose) | | |
| | 3^{rd} | Day | Prednisolone 100 mg IV | Diphenhydramine | Paracetamol |
| | epcoritamab | 15* | (or equivalent including | 50 mg IV or oral | (acetaminophen) |
| | administration | | oral dose) | (PO) (or equivalent) | 650 to 1000 mg PO |
| | (1^{st} full dose) | | | | (or equivalent) |
| | | Day | Prednisolone 100 mg IV | | |
| | | 16 | (or equivalent including | | |
| | | | oral dose) | | |
| | | Day | Prednisolone 100 mg IV | | |
| | | 17 | (or equivalent including | | |
| | | | oral dose) | | |
| | | Day | Prednisolone 100 mg IV | | |
| | | 18 | (or equivalent including | | |
| | | | oral dose) | | |
| | 4^{th} | Day | Prednisolone 100 mg IV | Diphenhydramine | Paracetamol |
| | epcoritamab | 22* | (or equivalent including | 50 mg IV or oral | (acetaminophen) |
| | administration | | oral dose) | (PO) (or equivalent) | 650 to 1000 mg PO |
| | (2^{nd} full dose) | | | | (or equivalent) |
| | | Day | Prednisolone 100 mg IV | | |
| | | 23 | (or equivalent including | | |
| | | | oral dose) | | |
| | | Day | Prednisolone 100 mg IV | | |
| | | 24 | (or equivalent including | | |
| | | | oral dose) | | |
| | | Day | Prednisolone 100 mg IV | | |
| | | 25 | (or equivalent including | | |
| | | | oral dose) | | |
| Cycle | 5^{th} | Day | If CRS ≥ grade 2 occurs | Optional, based on | Optional based on |
| 2 | epcoritamab | 29* | following the 4^{th} | prior occurrence of | prior occurrence of |
| | administration | | epcoritamab | injection site | injection site |
| | (3^{rd} full dose) | | administration, 4-day | reaction | reaction |
| | | Day | consecutive | | |
| | | 30 | corticosteroid | | |
| | | Day | administration is | | |
| | | 31 | continued in Cycle 2 until | | |
| | | Day | an epcoritamab dose is | | |
| | | 32 | given that does not result | | |
| | | | in subsequent CRS. | | |

*30 minutes-2 hours prior to administration of epcoritamab.
**Refer to Table 6 for corticosteroid dose equivalents.

TABLE 6
Corticosteroid Dose Equivalents - Conversion Table

| | |
|---|---|
| Glucocorticoid | Approximate equivalent dose (mg) |

Short-acting

| | |
|---|---|
| Cortisone (PO) | 500 |
| Hydrocortisone (IV or PO) | 400 |

Intermediate-acting

| | |
|---|---|
| Methylprednisolone (IV or PO) | 80 |
| Prednisolone (PO) | 100 |
| Prednisone (IV or PO) | 100 |
| Triamcinolone (IV) | 80 |

Long-acting

| | |
|---|---|
| Betamethasone (IV) | 15 |
| Dexamethasone (IV or PO) | 15 |

### Supportive Care for Cytokine Release Syndrome

CRS is graded according to the ASTCT grading for CRS (Tables 7 and 8), and for treatment of CRS, subjects should receive supportive care. Supportive care can include, but is not limited to,
Infusion of saline
Systemic glucocorticosteroid, antihistamine, antipyrexia
Support for blood pressure (vasopressin, vasopressors)
Support for low-flow and high-flow oxygen and positive pressure ventilation
Monoclonal antibody against IL-6R, e.g., IV administration of tocilizumab
Monoclonal antibody against IL-6, e.g., IV siltuximab if not responding to repeated tocilizumab.
Administration of Anakinra
Blood product support, analgesics, skin, and mouth care, etc., should be according to local guidelines and the investigator's discretion.

TABLE 7
Grading and Management of Cytokine Release Syndrome
Harmonized definitions and grading criteria for CRS, per the American Society
for Transplantation and Cellular Therapy (ASTCT), formerly American Society
for Blood and Marrow Transplantation, (ASBMT), are presented below.
Grading of Cytokine Release Syndrome

| CRS | | | | | |
|---|---|---|---|---|---|
| parameter | Grade 1 | Grade 2 | Grade 3 | Grade 4 | Grade 5 |
| Fever¹ | ≥38.0° C. | ≥38.0° C. | ≥38.0° C. | ≥38.0° C. | Death due |
| With | None | Not requiring | Requiring 1 | Requiring ≥2 | to CRS in |
| hypotension | | vasopressors | vasopressor | vasopressors | which |
| | | | with or | (excluding | another |
| | | | without | vasopressin) | cause is not |
| | | | vasopressin | | the |
| And/or | None | Requiring | Requiring | Requiring | principle |
| hypoxia² | | low-flow | high-flow | positive | factor |
| | | (□6 L/minute) | (>6 L/minute) | pressure | leading to |
| | | nasal cannula | nasal | ventilation³ | this |
| | | or blow-by | cannula, | (eg, CPAP, | outcome |
| | | | facemask, | BiPAP, | |
| | | | nonrebreather | intubation and | |
| | | | mask, or | mechanical | |
| | | | venturi mask | ventilation) | |

Abbreviations: BiPAP, Bilevel positive airway pressure; CPAP, continuous positive airway pressure; CRS, cytokine release syndrome; IV, intravenous.
Note:
organ toxicities or constitutional symptoms associated with CRS may be graded according to CTCAE but they do not influence CRS grading.
¹Fever is defined as temperature ≥38.0° C. not attributable to any other cause, with or without constitutional symptoms (eg, myalgia, arthralgia, malaise). In subjects who have CRS receiving antipyretics, anticytokine therapy, and/or corticosteroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS. Both systolic blood pressure and mean arterial pressure are acceptable for blood pressure measurement. No specific limits are required, but hypotension should be determined on a case-by-case basis, accounting for age and the subject's individual baseline, i.e., a blood pressure that is below the normal expected for an individual in a given environment.
³Intubation of a subject without hypoxia for the possible neurologic compromise of a patent airway alone or for a procedure is not by definition grade 4 CRS.
Source: Adapted from Lee et al., Biol Blood Marrow Transplant 2019; 25: 625-638

TABLE 8
Grading and Management of Cytokine Release Syndrome

| CRS grade | Management |
|---|---|
| 1 | Fever: Patients with a new fever should be admitted to the hospital if not already. Investigate |
| | for infection and rapidly startup broad-spectrum antibiotics. Continuation of antibiotic therapy |
| | is recommended until and potential neutropenia resolve. Constitutional symptoms may be |
| | helped by NSAIDs. |
| | Tocilizumab: No*. |
| | Steroids: No. |
| 2 | Fever: As per grade 1. |
| | Hypotension: Immediate clinical evaluation and intervention is warranted. At the first |
| | confirmed decrease ≥20% from baseline systolic, diastolic or mean arterial pressure or |
| | evidence of worsening perfusion, administer an IV fluid bolus (20 mL/kg up to 1 L). Consider a |
| | vasopressor and administer no later than after the 3^{rd} IV fluid bolus due the vasodilatation and |
| | capillary leak associated with CRS. |
| | Hypoxia: Consider X-ray or CT-scan if hypoxic and/or tachypneic. Administer oxygen by low- |
| | flow nasal cannula (≤6 L/min) or blow-by. |
| | Tocilizumab: No* (yes, if the patient has comorbidities^{†}). |
| | Steroids: No (consider, if the patient has comorbidities^{‡}). |
| 3 | Fever: As per grade 1. |
| | Hypotension: Immediate clinical evaluation and intervention is warranted. Administer a |
| | vasopressor (norepinephrine), with or without vasopressin, as most patients with CRS have |
| | peripheral vasodilation. |
| | Hypoxia: Administer oxygen by high-flow nasal cannula (>6 L/min), facemask, non-breather |
| | mask, or Venturi mask. |
| | Tocilizumab: Yes^{†}. |
| | Steroids: Consider^{‡}. |
| 4 | Fever: As per grade 1. |
| | Hypotension: Immediate clinical evaluation and intervention is warranted. Administer at least 2 |
| | vasopressors, with or without vasopressin, as most patients with CRS have peripheral |
| | vasodilation. |
| | Hypoxia: Positive pressure (e.g. CPAP, BiPAP, intubation, and mechanical ventilation). |
| | Tocilizumab: Yes^{†}. |
| | Steroids: Yes^{‡}. |

*Consider intervening earlier in specific cases. For example, an elderly patient with prolonged fever (>72 hours) or very high fever (>40.5° C./104.9° F.) may not tolerate the resulting sinus tachycardia as well as a younger patient, so tocilizumab may be indicated.
^{†}Tocilizumab (anti-IL-6R) remains the only first-line anticytokine therapy approved for CRS. If there is no improvement in symptoms within 6 hours, or if the patient starts to deteriorate after initial improvement, a second dose of tocilizumab should be administered along with a dose of corticosteroids. For patients being refractory to tocilizumab (3 administrations), additional anticytokine therapy such as siltuximab (anti-IL-6) or anakinra (anti-IL-1R) may be considered. However, such use is entirely anecdotal and, as such, is entirely at the discretion of the treating physician.
^{‡}Consider dexamethasone over methylprednisolone due to improved CNS penetration even in absence of neurotoxicity, as high-grade CRS is correlated with risk of concurrent or subsequent ICANS. If concurrent ICANS is observed, dexamethasone should be preferred.
Source: (Varadarajan I, Kindwall-Keller T L, Lee D W (2020). Management of cytokine release syndrome, In: Chimeric antigen receptor T-cell therapies for cancer (Chapter 5). Elsevier 2020)

### Tumor Lysis Syndrome Prevention and Management

For prophylactic treatment of tumor lysis syndrome, subjects receive uric acid reducing agents prior to the administration of epcoritamab, with allopurinol given at least 72 hours prior to the first dose of epcoritamab and rasburicase initiated prior to starting epcoritamab. Increased oral hydration should be received prior to the first dose and is maintained during dosings. Reassessment of the subject's TLS risk category is performed prior to subsequent doses.

### Study Assessments

### Bone Marrow Assessment

A fresh bone marrow aspirate is obtained at screening (i.e., within 21 days prior to Cycle 1 Day 1) and at the time of complete response (CR) or when clinically indicated. A fresh bone marrow biopsy is obtained at screening and at the time of CR or nodular partial response (PR) (nPR) or when clinically indicated. Bone marrow evaluations include morphological examination and either flow cytometry or immunohistochemistry.

### Radiographic Assessments

For RS, a ^{18F}-FDG-PET CT (or CT/MRI and FDG-PET when PET CT not available) must be performed at screening (ie, within 3 weeks prior to the first dose of GEN3013). For subjects with FDG-avid tumors at screening, all subsequent disease assessments will be performed with FDG-PET CT using the 5-point scale (Barrington et al., 2014). For subjects with non-avid or variably FDG-avid tumors, CT scan with IV contrast of neck/chest/abdomen/pelvis/additional known lesions will be performed. The CT component of the PET CT may be used in lieu of a standalone CT/MRI, only if the CT component is of similar diagnostic quality as a contrast enhanced CT performed without PET. If contrast enhanced PET CT is not available, a standalone diagnostic CT/MRI and a standard FDG-PET should be performed. If independent CT and PET scanners are used, and the subject is receiving both scans on the same day, the PET must be performed prior to the CT with IV contrast as to not compromise PET results. The PET CT acquisition methodology (eg, administration of intravenous contrast) should remain consistent between screening and subsequent assessments for any given subject

Imaging assessment schedules in both the dose escalation and expansion are performed as detailed in the Visit Assessment Schedules (Section 1). A CT scan with contrast is the recommended imaging modality. MRI may be used only if CT with contrast is medically contraindicated or if the frequency of CT scans exceeds local standards.

MRI may be used to evaluate sites of disease that cannot be adequately imaged using CT (in cases where MRI is desirable, the MRI must be obtained at screening and at all subsequent response evaluations). For all other sites of disease, MRI studies do not replace the required neck, chest, abdomen, and pelvic CT scans.

Additional imaging assessments may be performed at any time during the trial at the investigator's discretion to support the efficacy evaluations for a subject as necessary. Clinical suspicion of disease progression at any time requires a physical examination and imaging assessments to be performed promptly rather than waiting for the next scheduled imaging assessment.

### Minimal Residual Disease (MRD) Assessment

MRD is assessed in the blood by flow cytometry and next generation sequencing. After start of treatment, blood samples are requested at the fixed time points and at time of CR. As an exploratory analysis, when a subject reaches a CR, a portion of the aspirate collected to confirm CR is used to assess MRD.

### Disease Response and Progressive Disease Assessment

Tumor response according to imaging assessment is performed to inform decisions on continuation of treatment. Response assessment is completed according to Lugano criteria (Cheson et al., 2014, J Clin Oncol 32, 3059-3068) Table 2. Since local palliative radiotherapy on non-target lesions is permitted, if given during the trial, these lesions should no longer be included in the response assessment.

Lugano criteria (Cheson et al., 2014)Target and Non-target Lesions

Target lesions should consist of up to six of the largest dominant nodes, nodal masses, or other lymphomatous lesions that are measurable in two diameters and should preferably be from different body regions representative of the subject's overall disease burden, including mediastinal and retroperitoneal disease, where applicable. At baseline, a measurable node must be greater than 15 mm in longest diameter (LDi). Measurable extranodal disease may be included in the six representative target lesions. At baseline, measurable extranodal lesions should be greater than 10 mm in LDi. All other lesions (including nodal, extranodal, and assessable disease) should be followed as non-target lesions (eg, cutaneous, GI, bone, spleen, liver, kidneys, pleural or pericardial effusions, ascites, bone, bone marrow).

### Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease (PD). The LDi and smallest diameter (SDi) are no longer needed to determine progression.

Endpoint definitions are as follows:

Overall response rate (ORR), is defined as the proportion of subjects who achieve a response of PR or CR, prior to initiation of subsequent therapy.

Time to response (TTR), is defined among responders, as the time between first dose of epcoritamab and the initial documentation of PR or CR.

Duration of response (DOR), is defined among responders, as the time from the initial documentation of PR or CR to the date of disease progression or death, whichever occurs earlier.

Progression-free survival (PFS), is defined as the time from the first dosing date of epcoritamab and the date of disease progression or death, whichever occurs earlier.

Overall survival (OS), is defined as the time from the first dosing date of epcoritamab and the date of death.

MRD negativity rate, is defined as the proportion of subjects with at least 1 undetectable MRD result according to the specific threshold, prior to initiation of subsequent therapy.

Clinical Safety Assessments Safety will be assessed by measuring adverse events, laboratory test results, ECGs, vital

sign measurements, physical examination findings, and ECOG performance status. Also assessed are immune effector cell-associated neurotoxicity syndrome (e.g., as described by Lee et al., Biol Blood Marrow Transplant 2019; 25:625-638), constitutional symptoms (B symptoms), tumor flare reaction, and survival.

### Immunophenotyping Analyses

Absolute B and T-cell counts are determined in fresh whole blood using flow cytometry to monitor changes associated with epcoritamab treatment. The T-cell activation and exhaustion phenotype is evaluated using flow cytometry and markers in order to evaluate the association of such markers with drug target engagement, treatment efficacy, and/or safety of epcoritamab. Additional immunophenotype of circulating immune cells (e.g., levels of regulatory T-cells which can suppress T-cell function) are determined in fresh whole blood using flow cytometry to evaluate the association of such markers with T-cell activation/exhaustion phenotype, subject response, and epcoritamab's MOA.

### Cytokine and Endothelial Activation Marker Analyses

Since T-cell activation following initial epcoritamab administrations may lead to cytokine release causing CRS, cytokine levels are closely monitored. The levels of cytokines, such as IL-2, IL15, IL-6, IL-8, IL-10, IFNγ, and/or TNFα, are measured in plasma samples using an array based ligand binding assay. Additional cytokines may also be determined to evaluate the association of such markers with treatment-emergent AEs and outcome to epcoritamab.

### Preliminary Results:

The first patient was enrolled on Nov. 17, 2021.

Data cutoff on Jul. 15, 2022:

Ten patients with RS (median age, 69.5 y; range, 53-79) had received epcoritamab 48 mg with a follow-up of ≥12 wk. The median time from RS diagnosis to first dose of epcoritamab was 0.03 years (range, 0.0-01). Prior therapies for RS included rituximab, cyclophosphamide, doxorubicin, vincristine, and prednisone (R-CHOP) rituximab, dexamethasone, cytarabine, and cisplatin (R-DHAP) and venetoclax plus dose-adjusted rituximab, etoposide, prednisone, vincristine, cyclophosphamide, and doxorubicin (VR-EPOCH) and 50% of patients received epcoritamab as first-line therapy for RS. Median treatment duration was 2.5 mo (range, 0.5-6.5), with 5 (50%) patients receiving ongoing treatment. The most common related treatment-emergent AEs (TEAEs) of any grade were CRS (90%; 30% grade 1, 60% grade 2), anemia (30%), diarrhea (40%), hypophosphatemia (10%), injection-site reaction (30%), and thrombocytopenia (30%). Notable grade 3-4 TEAEs included neutropenia (n=4; 2 patients grade 3, 2 patients grade 4), anemia (n=2), and COVID-19 (n=2). Most CRS events were associated with the first full dose of epcoritamab. All CRS events resolved (median time to resolution, 3 d), no patients discontinued treatment due to CRS, and 7 patients received tocilizumab. No cases of ICANS were observed. Clinical tumor lysis syndrome (grade 2) occurred in 1 patient and resolved within 3 d. No patients discontinued treatment due to AEs. Two patients died due to disease progression. Antitumor activity was observed early (majority of responses seen at the first [wk 6] assessment), with an overall response rate of 60% and a complete response rate of 50%.
Data cutoff on Sep. 8, 2022 (efficacy)/Sep. 16, 2022 (safety):
A total of ten patients in the Richter's cohort had been dosed with 48 mg epcoritamab epcoritamab and were response evaluable. Median duration of treatment was 3.5 months (range: 0.5-9.3). Median 28-day cycles of epcoritamab was 4 (range 1-11). The main patient characteristics are provided below in tables 9 and 10, and the treatment history is provided in tables 11 and 12.

TABLE 9
Patient characteristics

| CLL Characteristic, n (%) | Total N = 10 |
|---|---|
| IGHV unmutated^{a} | 8 (80) |
| TP53 mutation^{b} | 5 (50) |
| NOTCH1 mutation^{c} | 2 (20) |
| FISH | |
| Trisomy 12^{d} | 1 (10) |
| Del17p^{e} | 3 (30) |
| Del11q^{f} | 3 (30) |
| Del13q^{g} | 4 (40) |

Data for CLL characteristics were obtained from local laboratories.
^{a}IGHV mutation status unknown for 2 patients.
^{b}TP53 mutation status unmutated for 4 patients and unknown for 1 patient.
^{c}NOTCH1 mutation status unmutated for 4 patients and unknown for 4 patients.
^{d}Trisomy 12 status negative for 8 patients and unknown for 1 patient.
^{e}Del17p status negative for 7 patients.
^{f}Del11q status negative for 7 patients.
^{g}Del13q status negative for 4 patients and unknown for 2 patients

TABLE 10
Patient characteristics

| RS Characteristic | N = 10 | |
|---|---|---|
| Median age, y (range) | 70 | (53-79) |
| Male, n (%) | 7 | (70) |
| Ann Arbor stage, n (%) | | |
| IE | 1 | (10) |
| II | 1 | (10) |
| III | 3 | (30) |
| IV | 5 | (50) |
| Elevated lactate dehydrogenase, n (%) | 8 | (80) |
| Cell of origin, n (%)^{a} | | |
| Germinal center B-cell | 1 | (10) |
| Non-germinal center/Activated B-cell | 6 | (60) |

Data cutoff: Sep. 16, 2022.
^{a}Cell of origin was unknown for 3 patients.

TABLE 11
Treatment history

| | Total | |
|---|---|---|
| Characteristic of Prior CLL Therapy | N = 10 | |
| Median time from initial CLL diagnosis to | 12 | (2.6-24.0) |
| first dose, y (range) | | |
| Median number of prior lines of therapy | 3 | (0-7) |
| for CLL (range) | | |
| Prior CLL/SLL therapy, n (%) | 7 | (70) |
| Chemoimmunotherapies | 7 | (70) |
| Targeted agents | 6 | (60) |
| BCL2 inhibitor | 5 | (50) |
| BTK inhibitor | 5 | (50) |
| CAR T-cell therapy | 1 | (10) |
| Median time from last CLL treatment to | 12 | (0.2-61.8) |
| first dose, mo (range) | | |

Data cutoff: Sep. 16, 2022.

TABLE 12
Treatment history

| | Total | |
|---|---|---|
| Characteristic of Prior RS-DLBCL Therapy | N = 10 | |
| 1 prior line of RS-DLBCL therapy, n (%) | 5 | (50) |
| R-CHOP | 3 | (30) |
| No response | 2 | (20) |
| R-DHAP | 1 | (10) |
| No response | 1 | (10) |
| VR-EPOCH^{a} | 1 | (10) |
| Median time from disease transformation | 3.4 | (0.5-21.4) |
| to first dose, mo (range) | | |
| Median time from end of last line of RS-DLBCL | 2 | (0.5-5.4) |
| therapy to first dose, mo (range) | | |

^{a}Response to VR-EPOCH unknown.
R-CHOP, rituximab, cyclophosphamide, doxorubicin, vincristine, and prednisone; R-DHAP, rituximab, dexamethasone, cytarabine, and cisplatin; VR-EPOCH, venetoclax plus dose-adjusted rituximab, etoposide, prednisone, vincristine, cyclophosphamide, and doxorubicin.

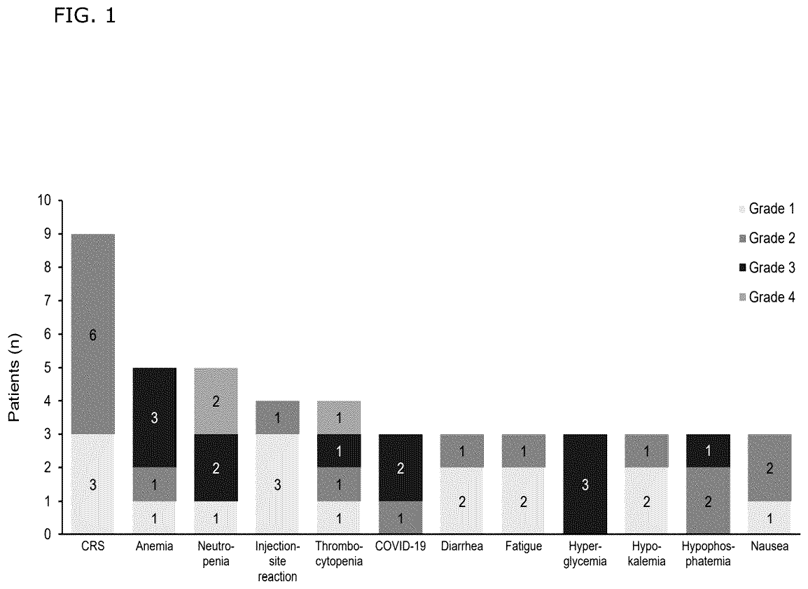

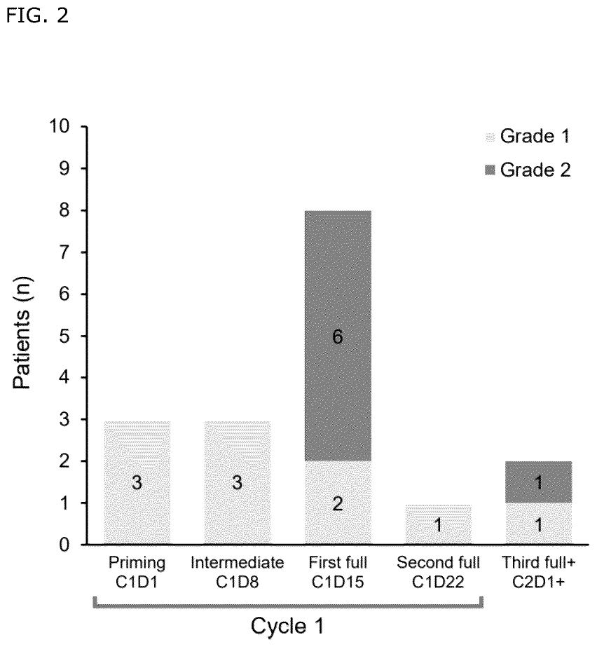

Most common treatment-emergent adverse events are shown in FIG. 1. Adverse events were primarily low grade, including CRS; no ICANS events were observed. One case of clinical tumor lysis syndrome (grade 2) was observed; it was resolved in 3 days. One grade 5 (fatal) treatment emergent adverse event (TEAE) was observed: general physical health deterioration in the setting of progressive disease; unrelated to Epcoritamab). Six patients experienced dose delay due to TEAE. No TEAE led to discontinuation.
CRS events were recorded as shown in Table 13. CRS events by dosing period is shown in FIG. 2. Occurrence of CRS was predictable, with most cases following the first full dose of Epcoritamab. No grade 3 or higher CRS events were observed. All CRS events were resolved, and none led to discontinuation of treatment.

TABLE 13
CSR events

| | Total, N = 10 | |
|---|---|---|
| CRS, n (%)^{a} | 9 | (90) |
| Grade 1 | 3 | (30) |
| Grade 2 | 6 | (60) |
| CRS resolution, n/n (%) | 9/9 | (100) |
| Median time to onset after first full dose, h (range) | 12.5 | (8-394) |
| Median time to resolution, d (range)^{b} | 3 | (2-9) |
| Treated with tocilizumab, n (%) | 7 | (70) |

Data cutoff: Sep. 16, 2022.
^{a}Graded by Lee et al 2019 criteria.
^{b}Median is Kaplan-Meier estimate based on longest CRS duration in patients with CRS.

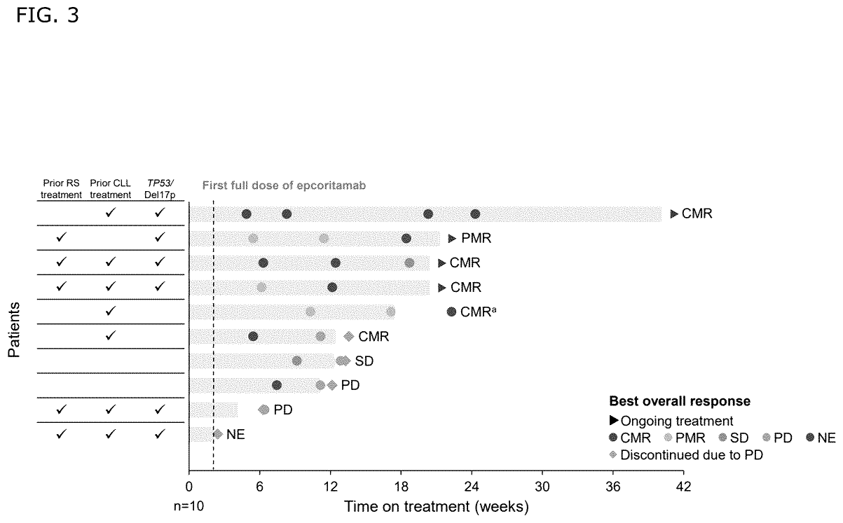

Depth and duration of the responses are shown in FIG. 3. Median time to response was 1.3 month (range: 1.1-2.4 months). Median time to complete response was 1.4 month (range: 1.1-2.8 months). Best overall response data is provided below in Table 14.

TABLE 14
Best overall response

| | Total Efficacy Evaluable |
|---|---|
| Response, n (%)^{a} | N = 10 |
| Overall response^{b} | 6 (60) |
| Complete metabolic response (CMR) | 5 (50) |
| Partial metabolic response (PMR) | 1 (10) |
| Stable disease | 1 (10) |
| Progressive disease | 2 (20) |

Data cutoff: Sep. 8, 2022. Median follow-up: 4.9 mo (range, 0.6-9.3).
^{a}Based on modified response-evaluable population, defined as patients with ≥1 target lesion at baseline and ≥1 postbaseline response evaluation and/or patients who died within 60 d of first dose.
^{b}Response assessment according to Lugano 2014 criteria.
^{c}Patient stopped treatment on C1D15 due to progression and did not receive any scans.

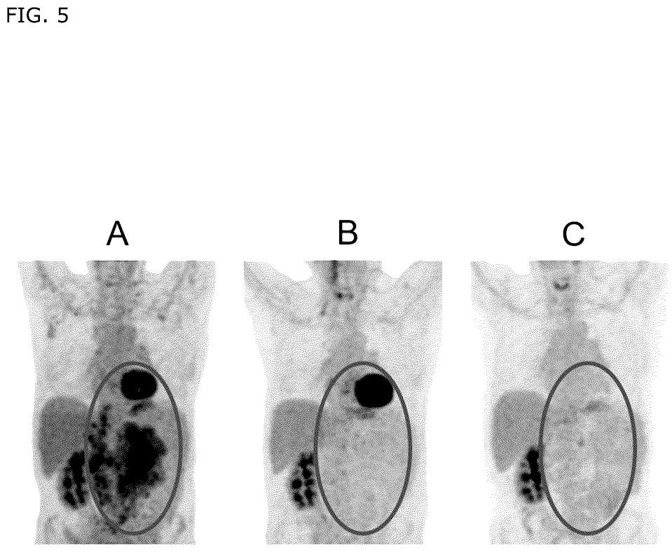

Tumor reduction from baseline (best overall response) is shown in FIG. 4.
FIG. 5 shows a clinical case study of RS-DLBCL.
Patient history:
76-year-old male
Diagnosed with SLL in Jul. 2019, started on ibrutinib
Transformed to RS-DLBCL in October 2020
RS-DLBCL treated with 3 cycles of
R-CHOP, mixed response
   Epcoritamab treatment:
First dose: SPD=105 cm²
CR at week 6, 12, 17, 23, 36, 48, 62, 76; DS=1, sum of the product of the diameters (SPD)=2.8 cm²
Patient has been in sustained CR for over 70 weeks and is still on treatment (last dose C22D1)
   As of 15 Aug. 2023, 30 subjects had been treated with at least one dose of epcoritamab. The observed overall response rate and complete showed similar overall response rate and complete metabolic response rate were similar with results from the September 2022 cut-off with a consistent, manageable and tolerable safety profile.

### Conclusion:

Based on data from a data cut-off of Sep. 8, 2022 (efficacy)/Sep. 16, 2022 (safety), the following conclusion was drawn: In patients with RS, SC epcoritamab demonstrated a manageable safety profile with low-grade CRS events. Most CSR events occurred in cycle 1 after the first full dose of epcoritamab. All resolved and none led to treatment discontinuation. This safety profile was consistent with previous reports of epcoritamab monotherapy, and no new safety signals were reported. Preliminary efficacy findings show that SC epcoritamab has encouraging single-agent activity in RS-DLBCL, with high overall and complete response rates observed and the majority of responses seen at the first (wk 6) assessment.

Based on data from a data cut-offs of September 2022, the conclusion remained favorable: Epcoritamab demonstrated promising activity with high overall response and CMR rates and a tolerable safety profile
Overall response rate (ORR): 60%; Complete metabolic response rate (CMR): 50%
Only low-grade cytokine release syndrome (CRS); all resolved
No ICANS events
One case of grade 2 CTLS, which resolved in 3 days
No discontinuations due to treatment emergent adverse events (TEAEs).
   As of Aug. 15, 2023 epcoritamab continued to show a high preliminary ORR and CR rates with a manageable and tolerable safety profile. This demonstrates that epcoritamab has the potential to become an effective, convenient, and tolerable treatment option with a favorable benefit risk profile for patients with RS.

TABLE 13
Listing of Sequences

| SEQ | | |
|---|---|---|
| ID | Description | Sequence |
| 1 | huCD3 VH CDR1 | GFTFNTYA |
| 2 | huCD3 VH CDR2 | IRSKYNNYAT |
| 3 | huCD3 VH CDR3 | VRHGNFGNSYVSWFAY |
| 4 | huCD3 VL CDR1 | TGAVTTSNY |
| — | huCD3 VL CDR2 | GTN |
| 5 | huCD3 VL CDR3 | ALWYSNLWV |
| 6 | huCD3 VH1 | EVKLVESGGGLVQPGGSLRLSCAASGFTFNTYAMNWVRQAPGKG |
| | | LEWVARIRSKYNNYATYYADSVKDRFTISRDDSKSSLYLQMNNLK |
| | | TEDTAMYYCVRHGNFGNSYVSWFAYWGQGTLVTVSS |
| 7 | huCD3 VL1 | QAVVTQEPSFSVSPGGTVTLTCRSSTGAVTTSNYANWVQQTPGQ |
| | | AFRGLIGGTNKRAPGVPARFSGSLIGDKAALTITGAQADDESIYFC |
| | | ALWYSNLWVFGGGTKLTVL |
| 8 | VH CD20-7D8 | GFTFHDYA |
| | CDR1 | |
| 9 | VH CD20-7D8 | ISWNSGTI |
| | CDR2 | |
| 10 | VH CD20-7D8 | AKDIQYGNYYYGMDV |
| | CDR3 | |
| 11 | VL CD20-7D8 | QSVSSY |
| | CDR1 | |
| — | VL CD20-7D8 | DAS |
| | CDR2 | |
| 12 | VL CD20-7D8 | QQRSNWPIT |
| | CDR3 | |
| 13 | VH CD20-7D8 | EVOLVESGGGLVQPDRSLRLSCAASGFTFHDYAMHWVRQAPGKG |
| | | LEWVSTISWNSGTIGYADSVKGRFTISRDNAKNSLYLQMNSLRAE |
| | | DTALYYCAKDIQYGNYYYGMDVWGQGTTVTVSS |
| 14 | VL CD20-7D8 | EIVLTQSPATLSLSPGERATLSCRASQSVSSYLAWYQQKPGQAPRL |
| | | LIYDASNRATGIPARFSGSGSGTDFTLTISSLEPEDFAVYYCQQRS |
| | | NWPITFGQGTRLEIK |
| 15 | IgG1 heavy chain | ASTKGPSVFPLAPSSKSTSGGTAALGCLVKDYFPEPVTVSWNSGA |
| | constant region- | LTSGVHTFPAVLQSSGLYSLSSVVTVPSSSLGTQTYICNVNHKPSN |
| | WT | TKVDKRVEPKSCDKTHTCPPCPAPELLGGPSVFLFPPKPKDTLMISR |
| | (amino acids | TPEVTCVVVDVSHEDPEVKFNWYVDGVEVHNAKTKPREEQYNSTY |
| | positions 118-447 | RVVSVLTVLHQDWLNGKEYKCKVSNKALPAPIEKTISKAKGQPREP |
| | according to EU | QVYTLPPSREEMTKNQVSLTCLVKGFYPSDIAVEWESNGQPENNY |
| | numbering). | KTTPPVLDSDGSFFLYSKLTVDKSRWQQGNVFSCSVMHEALHNH |
| | CH3 region italics | YTQKSLSLSPG |
| 16 | IgG1-LFLEDA heavy | ASTKGPSVFPLAPSSKSTSGGTAALGCLVKDYFPEPVTVSWNSGA |
| | chain constant | LTSGVHTFPAVLQSSGLYSLSSVVTVPSSSLGTQTYICNVNHKPSN |
| | region (amino acids | TKVDKRVEPKSCDKTHTCPPCPAPEFEGGPSVFLFPPKPKDTLMIS |
| | positions 118-447 | RTPEVTCVVVAVSHEDPEVKFNWYVDGVEVHNAKTKPREEQYNS |
| | according to EU | TYRVVSVLTVLHQDWLNGKEYKCKVSNKALPAPIEKTISKAKGQPR |
| | numbering). | EPQVYTLPPSREEMTKNQVSLTCLVKGFYPSDIAVEWESNGQPEN |
| | | NYKTTPPVLDSDGSFFLYSKLTVDKSRWQQGNVFSCSVMHEALH |
| | | NHYTQKSLSLSPG |
| 17 | IgG1 F405L | ASTKGPSVFPLAPSSKSTSGGTAALGCLVKDYFPEPVTVSWNSGA |
| | (amino acids | LTSGVHTFPAVLQSSGLYSLSSVVTVPSSSLGTQTYICNVNHKPSN |
| | positions 118-447 | TKVDKRVEPKSCDKTHTCPPCPAPELLGGPSVFLFPPKPKDTLMISR |
| | according to EU | TPEVTCVVVDVSHEDPEVKFNWYVDGVEVHNAKTKPREEQYNSTY |
| | numbering) | RVVSVLTVLHQDWLNGKEYKCKVSNKALPAPIEKTISKAKGQPREP |
| | | QVYTLPPSREEMTKNQVSLTCLVKGFYPSDIAVEWESNGQPENNY |
| | | KTTPPVLDSDGSFLLYSKLTVDKSRWQQGNVFSCSVMHEALHNH |
| | | YTQKSLSLSPG |
| 18 | IgG1-K409R | ASTKGPSVFPLAPSSKSTSGGTAALGCLVKDYFPEPVTVSWNSGA |
| | (amino acids | LTSGVHTFPAVLQSSGLYSLSSVVTVPSSSLGTQTYICNVNHKPSN |
| | positions 118-447 | TKVDKRVEPKSCDKTHTCPPCPAPELLGGPSVFLFPPKPKDTLMISR |
| | according to EU | TPEVTCVVVDVSHEDPEVKFNWYVDGVEVHNAKTKPREEQYNSTY |
| | numbering) | RVVSVLTVLHQDWLNGKEYKCKVSNKALPAPIEKTISKAKGQPREP |
| | | QVYTLPPSREEMTKNQVSLTCLVKGFYPSDIAVEWESNGQPENNY |
| | | KTTPPVLDSDGSFFLYSRLTVDKSRWQQGNVFSCSVMHEALHNH |
| | | YTQKSLSLSPG |
| 19 | IgG1-LFLEDA- | ASTKGPSVFPLAPSSKSTSGGTAALGCLVKDYFPEPVTVSWNSGA |
| | F405L (FEAL) | LTSGVHTFPAVLQSSGLYSLSSVVTVPSSSLGTQTYICNVNHKPSN |
| | (amino acids | TKVDKRVEPKSCDKTHTCPPCPAPEFEGGPSVFLFPPKPKDTLMIS |
| | positions 118-447 | RTPEVTCVVVAVSHEDPEVKFNWYVDGVEVHNAKTKPREEQYNS |
| | according to EU | TYRVVSVLTVLHQDWLNGKEYKCKVSNKALPAPIEKTISKAKGQPR |
| | numbering) | EPQVYTLPPSREEMTKNQVSLTCLVKGFYPSDIAVEWESNGQPEN |
| | | NYKTTPPVLDSDGSFLLYSKLTVDKSRWQQGNVFSCSVMHEALH |
| | | NHYTQKSLSLSPG |
| 20 | IgG1-LFLEDA- | ASTKGPSVFPLAPSSKSTSGGTAALGCLVKDYFPEPVTVSWNSGA |
| | K409R (FEAR) | LTSGVHTFPAVLQSSGLYSLSSVVTVPSSSLGTQTYICNVNHKPSN |
| | (amino acids | TKVDKRVEPKSCDKTHTCPPCPAPEFEGGPSVFLFPPKPKDTLMIS |
| | positions 118-447 | RTPEVTCVVVAVSHEDPEVKFNWYVDGVEVHNAKTKPREEQYNS |
| | according to EU | TYRVVSVLTVLHQDWLNGKEYKCKVSNKALPAPIEKTISKAKGQPR |
| | numbering) | EPQVYTLPPSREEMTKNQVSLTCLVKGFYPSDIAVEWESNGQPEN |
| | | NYKTTPPVLDSDGSFFLYSRLTVDKSRWQQGNVFSCSVMHEALH |
| | | NHYTQKSLSLSPG |
| 21 | IgG1 CH3 region | GQPREPQVYTLPPSREEMTKNQVSLTCLVKGFYPSDIAVEWESNG |
| | | QPENNYKTTPPVLDSDGSFFLYSKLTVDKSRWQQGNVFSCSVMH |
| | | EALHNHYTQKSLSLSPG |
| 22 | Constant region | GQPKAAPSVTLFPPSSEELQANKATLVCLISDFYPGAVTVAWKADS |
| | human lambda LC | SPVKAGVETTTPSKQSNNKYAASSYLSLTPEQWKSHRSYSCQVTH |
| | | EGSTVEKTVAPTECS |
| 23 | Constant region | RTVAAPSVFIFPPSDEQLKSGTASVVCLLNNFYPREAKVQWKVDN |
| | human kappa LC | ALQSGNSQESVTEQDSKDSTYSLSSTLTLSKADYEKHKVYACEVT |
| | | HQGLSSPVTKSFNRGEC |
| 24 | huCD3-LFLEDA- | EVKLVESGGGLVQPGGSLRLSCAASGFTFNTYAMNWVRQAPGKG |
| | F405L (FEAL) | LEWVARIRSKYNNYATYYADSVKDRFTISRDDSKSSLYLQMNNLK |
| | heavy chain | TEDTAMYYCVRHGNFGNSYVSWFAYWGQGTLVTVSSASTKGPSV |
| | | FPLAPSSKSTSGGTAALGCLVKDYFPEPVTVSWNSGALTSGVHTFP |
| | | AVLQSSGLYSLSSVVTVPSSSLGTQTYICNVNHKPSNTKVDKRVEP |
| | | KSCDKTHTCPPCPAPEFEGGPSVFLFPPKPKDTLMISRTPEVTCVV |
| | | VAVSHEDPEVKFNWYVDGVEVHNAKTKPREEQYNSTYRVVSVLT |
| | | VLHQDWLNGKEYKCKVSNKALPAPIEKTISKAKGQPREPQVYTLPP |
| | | SREEMTKNQVSLTCLVKGFYPSDIAVEWESNGQPENNYKTTPPVL |
| | | DSDGSFLLYSKLTVDKSRWQQGNVFSCSVMHEALHNHYTQKSLS |
| | | LSPG |
| 25 | huCD3 VL + CL light | QAVVTQEPSFSVSPGGTVTLTCRSSTGAVTTSNYANWVQQTPGQ |
| | chain | AFRGLIGGTNKRAPGVPARFSGSLIGDKAALTITGAQADDESIYFC |
| | | ALWYSNLWVFGGGTKLTVLGQPKAAPSVTLFPPSSEELQANKATL |
| | | VCLISDFYPGAVTVAWKADSSPVKAGVETTTPSKQSNNKYAASSY |
| | | LSLTPEQWKSHRSYSCQVTHEGSTVEKTVAPTECS |
| 26 | CD20-7D8-LFLEDA- | EVOLVESGGGLVQPDRSLRLSCAASGFTFHDYAMHWVRQAPGKG |
| | K409R (FEAR) | LEWVSTISWNSGTIGYADSVKGRFTISRDNAKNSLYLQMNSLRAE |
| | heavy chain | DTALYYCAKDIQYGNYYYGMDVWGQGTTVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKRVEPKS |
| | | CDKTHTCPPCPAPEFEGGPSVFLFPPKPKDTLMISRTPEVTCVVVA |
| | | VSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRE |
| | | EMTKNQVSLTCLVKGFYPSDIAVEWESNGQPENNYKTTPPVLDSD |
| | | GSFFLYSRLTVDKSRWQQGNVFSCSVMHEALHNHYTQKSLSLSP |
| | | G |
| 27 | CD20-7D8 VL + CL | EIVLTQSPATLSLSPGERATLSCRASQSVSSYLAWYQQKPGQAPRL |
| | light chain | LIYDASNRATGIPARFSGSGSGTDFTLTISSLEPEDFAVYYCQQRS |
| | | NWPITFGQGTRLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| 28 | Human CD3 | MQSGTHWRVLGLCLLSVGVWGQDGNEEMGGITQTPYKVSISGTT |
| | (epsilon) | VILTCPQYPGSEILWOHNDKNIGGDEDDKNIGSDEDHLSLKEFSE |
| | | LEQSGYYVCYPRGSKPEDANFYLYLRARVCENCMEMDVMSVATIVI |
| | | VDICITGGLLLLVYYWSKNRKAKAKPVTRGAGAGGRQRGQNKERP |
| | | PPVPNPDYEPIRKGQRDLYSGLNQRRI |
| 29 | Human CD20 | MTTPRNSVNGTFPAEPMKGPIAMQSGPKPLFRRMSSLVGPTQSFF |
| | | MRESKTLGAVQIMNGLFHIALGGLLMIPAGIYAPICVTVWYPLWGG |
| | | IMYIISGSLLAATEKNSRKCLVKGKMIMNSLSLFAAISGMILSIMDI |
| | | LNIKISHFLKMESLNFIRAHTPYINIYNCEPANPSEKNSPSTQYCYSI |
| | | QSLFLGILSVMLIFAFFQELVIAGIVENEWKRTCSRPKSNIVLLSAEE |
| | | KKEQTIEIKEEVVGLTETSSQPKNEEDIEIIPIQEEEEEETETNFPEPP |
| | | QDQESSPIENDSSP |

Bold and underlined are FE; A; L and R, corresponding to positions 234 and 235; 265; 405 and 409, respectively, said positions being in accordance with EU-numbering. In variable regions, said CDR regions that were annotated in accordance with IMGT definitions are underlined.

1. A method of treating Richter's syndrome (RS) in a human subject, the method comprising administering to the subject a bispecific antibody comprising:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose ranging from 12-60 mg in 28-day cycles,
wherein administration of the bispecific antibody continues at least until the subject exhibits a complete response (CR), a partial response (PR), or stable disease, or until progressive disease develops or unacceptable toxicity occurs.

2. The method of claim 1, wherein the bispecific antibody is administered at a dose of 24 mg.

3. The method of claim 1, wherein the bispecific antibody is administered at a dose of 48 mg.

4. The method of claim 1, wherein the bispecific antibody is administered once every week (weekly administration) for 2.5 28-day cycles.

5. (canceled)

6. The method of claim 4, wherein after the weekly administration, the bispecific antibody is administered once every two weeks (biweekly administration) for six 28-day cycles.

7. (canceled)

8. The method of claim 6, wherein after the biweekly administration, the bispecific antibody is administered once every four weeks.

9. The method of claim 4, wherein prior to administering the first weekly dose of 12-60 mg, a priming dose of the bispecific antibody is administered in cycle 1 of the 28-day cycles.

10. The method of claim 9, wherein the priming dose is administered two weeks prior to administering the first weekly dose of 12-60 mg.

11. The method of claim 9, wherein the priming dose is in the range of 0.05-0.35 mg.

12. The method of claim 9, wherein said priming dose is 0.16 mg or about 0.16 mg.

13. The method of claim 9, wherein after administering the priming dose and prior to administering the first weekly dose of 12-60 mg, an intermediate dose of the bispecific antibody is administered.

14. The method of claim 13, wherein the priming dose is administered on day 1 and the intermediate dose is administered on day 8 before the first weekly dose of 12-60 mg on days 15 and 22 of cycle 1.

15. The method of claim 13, wherein said intermediate dose is in the range of 0.6-1.2 mg.

16. The method of claim 13, wherein said intermediate dose is 0.8 mg or about 0.8 mg.

17. The method of claim 13, wherein the bispecific antibody is administered in 28-day cycles, wherein:
a) in cycle 1, a priming dose is administered on day 1, an intermediate dose on day 8, and a full dose of 12-60 mg on days 15 and 22;
b) in cycles 2-3, a full dose of 12-60 mg is administered on days 1, 8, 15, and 22;
c) in cycles 4-9, a full dose of 12-60 mg is administered on days 1 and 15; and
d) in cycle 10 and subsequent cycles, a full dose of 12-60 mg is administered on day 1.

18. The method of claim 17, wherein the full dose is 24 mg or about 24 mg.

19. The method of claim 17, wherein the full dose is 48 mg or about 48 mg.

20. The method of claim 1, wherein the bispecific antibody is administered subcutaneously.

21. The method of a claim 1, wherein the subject has clinical history of CLL/SLL with transformation toward aggressive lymphoma; e.g. of the DLBCL subtype.

22. The method of claim 1, wherein the Richter's syndrome is of the DLBCL subtype.

23. The method of claim 1, wherein the subject has received one or more, such as at least two, prior lines of therapy for Chronic Lymphocytic Leukemia (CLL) and/or for Small Lymphocytic Lymphoma (SLL).

24. The method of claim 23, wherein the prior lines of therapy for CLL and/or SLL comprise; (a) chemoimmunotherapy, (b) therapy with a targeted agent, such as BCL2 inhibitor or a BTK inhibitor, and/or (c) CAR T-cell therapy.

25-26. (canceled)

27. The method of claim 1, wherein the subject has received prior therapy for Richter's syndrome, such as prior therapy selected from:
i) Rituximab in combination with cyclophosphamide, doxorubicin, vincristine, and prednisone (R-CHOP),
ii) Rituximab in combination with dexamethasone, cytarabine, and cisplatin (R-DHAP), and
iii) Venetoclax in combination with rituximab, etoposide, prednisone, vincristine, cyclophosphamide, and doxorubicin (VR-EPOCH).

28. The method of claim 1, wherein the subject achieves a complete metabolic response or a partial metabolic response.

29. (canceled)

30. The method of claim 1, wherein the subject receives epcoritamab as first-line therapy for Richter's syndrome.

31-33. (canceled)

34. The method of claim 23, wherein the subject has refractory and/or relapsed Richter's syndrome after receiving the said prior therapies.

35. The method of claim 1, wherein the subject is treated with prophylaxis for cytokine release syndrome (CRS) and/or tumor lysis syndrome.

36-40. (canceled)

41. The method of claim 1, wherein the subject is administered premedication to reduce reactions to injections.

42-67. (canceled)

68. The method of claim 1, wherein:
(i) the first antigen-binding region comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

69. The method of claim 1, wherein:
(i) the first antigen-binding region comprises a VH region comprising the amino acid sequence of SEQ ID NO: 6, and the VL region comprising the amino acid sequence of SEQ ID NO: 7; and
(ii) the second antigen-binding region comprises a VH region comprising the amino acid sequence of SEQ ID NO: 13, and the VL region comprising the amino acid sequence of SEQ ID NO: 14.

70. The method of claim 1, wherein the first binding arm of the bispecific antibody is derived from a humanized antibody and comprises a λ light chain constant region comprising the amino acid sequence set forth in SEQ ID NO: 22 and/or the second binding arm of the bispecific antibody is derived from a human antibody and comprises a κ light chain constant region comprising the amino acid sequence set forth in SEQ ID NO: 23.

71-73. (canceled)

74. The method of claim 1, wherein the bispecific antibody is a full-length antibody with a human IgG1 constant region.

75-77. (canceled)

78. The method of claim 1, wherein the bispecific antibody comprises a first heavy chain and a second heavy chain, wherein
(i) in both the first and second heavy chains, the amino acids in the positions corresponding to positions L234, L235, and D265 in the human IgG1 heavy chain constant region of SEQ ID NO: 15 are F, E, and A, respectively, and
(ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

79. The method of claim 78, wherein the bispecific antibody comprises heavy chain constant regions comprising the amino acid sequences of SEQ ID NOs: 19 and 20.

80. The method of claim 1, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.

81. (canceled)

82. The method of claim 1, wherein the bispecific antibody is epcoritamab, or a biosimilar thereof.
